(19) United States
(12) Patent Application Publication
(10) Pub. No.: US 2026/0127966 A1
(43) Pub. Date: May 7, 2026

(54) SYSTEM AND METHOD FOR TRAFFIC CONTROL USING REAL-TIME CROSSING PREDICTION OF PEDESTRIANS

(71) Applicant: University of Central Florida Research Foundation, Inc., Orlando, FL (US)

(72) Inventors: Mohamed Abdel-Aty, Sanford, FL (US); Zubayer Islam, Orlando, FL (US); Ahmed Abdelrahman, Orlando, FL (US)

(21) Appl. No.: 19/378,012

(22) Filed: Nov. 3, 2025

Related U.S. Application Data
(60) Provisional application No. 63/715,387, filed on Nov. 1, 2024.

(51) Int. Cl. G08G 1/16; A61B 5/11; G08G 1/04; G08G 1/07

(52) U.S. Cl. CPC G08G 1/166; A61B 5/1128; G08G 1/04; G08G 1/07; A61B 2503/12

(57) ABSTRACT

A system controls intersection vehicle and pedestrian crossing signals using a camera to automatically detect when a pedestrian intends to cross a roadway at a crosswalk. The system automatically detects pedestrian presence, and uses trained AI to determine from pedestrian pose and location data whether the pedestrian intends to cross the roadway. On detection of the pedestrian's intent to cross, the system transmits a signal to the intersection controller corresponding to the pedestrian manual button signal, and causing the controller to initiate the pedestrian crossing phase. The system also monitors the presence of the pedestrian in the crosswalk, and extends the pedestrian crossing phase when needed, and also displays indicators directing vehicles to yield to pedestrians or make no turn on red where a pedestrian is detected in a crosswalk with potential conflict with a vehicle traffic signal.

### RELATED APPLICATION

This application claims the benefit of U.S. provisional application Ser. No. 63/715,387 filed on Nov. 1, 2024, which is herein incorporated by reference in its entirety.

This invention was made with Government support under Grant BED26-977-03 awarded by the Florida Department of Transportation (FDOT) and Grant BED26-977-03-562-6 awarded by the Florida Department of Transportation (FDOT). The Government has certain rights in this invention.

### FIELD OF THE INVENTION

This invention relates to monitoring or predicting movement of persons, especially pedestrians and other vulnerable road users (VRUs), e.g., bicyclists, scooter riders, etc., and more particularly to systems for monitoring pedestrian movement in the context of traffic signal efficiency and VRU safety and eventually incorporation in signal control systems.

### BACKGROUND OF THE INVENTION

Pedestrian safety is a global concern, particularly acute at intersections where pedestrian and vehicle paths commonly intersect. Enhancing pedestrian safety is paramount in pursuit of the Vision Zero initiative, which arms to eliminate traffic fatalities and severe injuries.

Elevating the safety of all vulnerable road users (VRUs), such as pedestrians and cyclists, is vital in the pursuit of Vision Zero. In the United States, traffic crashes resulted in 6,516 pedestrian fatalities in 2020, a 3.9% increase from 6272 pedestrian fatalities in 2019, and an estimated 54,769 pedestrians were injured. In 2020, the number of bicyclist fatalities reached 938, 26% of which occurred at intersections. This represented a 9% increase in bicyclist fatalities, up from 859 in 2019. Pedestrian fatalities represent 17% of all traffic fatalities, with an increase of 53% in 2018 compared to 2009. Florida ranks third in terms of pedestrian fatalities per capita in the U.S., and the Orlando-Kissimmee-Sanford area is fifth among the deadliest metropolitan regions, with 656 pedestrian deaths recorded between 2008 and 2017. To mitigate these alarming numbers, researchers are exploring preventive measures using advanced techniques to proactively gauge crash likelihood.

Enhancing the safety of vulnerable road users (VRUs) at intersections aims to eradicate traffic fatalities and severe injuries. The disregard of VRUs for designated signals, leading to crossing violations, is a significant issue, with studies showing that about half of VRUs at intersections fail to use the designated buttons for activating pedestrian signals, often due to unclear button placement. A major reason is pedestrian non-compliance with designated signals, leading to jaywalking. This appears to possibly be due to unclear button placement, insufficient signal timing, and distractions, which prevent pedestrians from activating signals or adhering to traffic rules. It also appears that, over half of pedestrians do not use the designated button to activate the pedestrian signal at intersections, and unclear button placement often leads to multiple buttons being pressed.

The fundamental reasons for VRU-vehicle conflicts at intersections, which are significant factors in severe accidents, are apparently that VRUs frequently fail to activate crossing signals by pressing the push button and often disregard signals when crossing when it is not their turn. Such behavior places them at risk of dangerous encounters with oncoming traffic. VRU violations and frequent disregard for compliance emerge as leading factors contributing to VRU conflicts at intersections.

Furthermore, it is desirable not only to enhance VRU safety but also to simultaneously maintain or enhance pedestrian signal performance. Improving signal performance while ensuring VRU safety is driven by the need to balance the critical aspects of urban mobility: the safety of VRUs and the efficiency of vehicular traffic flow. In modern urban centers, intersections are more than mere crossroads; they are pivotal points where the safety of VRUs such as pedestrians and cyclists intersect with the smooth functioning of traffic.

Enhancing pedestrian safety while improving pedestrian signal performance is important for balancing urban mobility's critical aspects: pedestrian safety and vehicular traffic flow efficiency. Traditional traffic signal systems often struggle with the challenge of maintaining VRU safety at intersections without impeding vehicular flow, leading to a compromise on either safety or efficiency.

It is desirable to provide safe and ample crossing experiences for VRUs while reducing unnecessary vehicle delays caused by false calls during the pedestrian phase. This not only enhances the safety and experience of VRUs but also contributes to a more fluid and less congested traffic system. Intersections are vital points in urban centers where pedestrian safety and traffic functionality converge.

Traditional traffic signal systems often fail to maintain pedestrian safety without compromising traffic flow, resulting in inefficiencies. Studies of pedestrian safety at intersections using computer vision and machine learning generally lack integration of their contributions in a real-time system. Additionally, no sophisticated real-time system has been developed that can be deployed with real-world scenarios verifications to ensure their applicability and reliability.

Many studies have considered some of these issues but there are two main gaps in what is addressed:
At a typical four-leg intersection, there are four waiting areas that each serve two crosswalks but the focus is only on deciding whether the VRU will cross or not, without specifying which crosswalk it is intended to go through.
There is a lack of real-time processing in predicting VRU crossing intention. Real-time processing stands as an important element in the development of a practical and intelligent system suitable for deployment at intersections.

Existing systems therefore have at least the following deficiencies:
The systems do not work in real-time.
There is no multi-directional intention prediction where more than one crosswalk is involved.
They result in increased delay for vehicles.
There is pedestrian non-compliance for longer waiting times.
They provide insufficient safety for pedestrians at crossings.

### SUMMARY OF THE INVENTION

It is therefore an object of the present invention to overcome the deficiencies in the prior state-of-the-practice.

Addressing this, a real-time system employing computer vision technology and artificial intelligence predicts the crossing intentions of VRUs. This system, which utilizes video feeds from four strategically positioned cameras at four-legged intersections and employs YOLOv8 and OC-SORT for accurate detection, tracking, and pose estimation, has shown outstanding results in predicting crossing intentions for various types of VRUs during both daytime and nighttime. The system achieves real-time processing, predicting VRU crossing intentions with an average accuracy of 94.67%, and average video processing of 33 FPS (frames per second). This system facilitates the automatic activation of pedestrian signals, eliminating the need for push-buttons and reducing false calls, thereby enhancing signal performance. The predictive system reduces cycle lengths by 15% while optimizing pedestrian and vehicle timing without compromising safety. Additionally, the inclusion of an extension checker aids in monitoring VRUs on the crosswalk, ensuring sufficient time to cross safely and comfortably. The system also predicts the potential of conflicts between pedestrians and right-turning vehicles in the near crosswalk during the red-signal phase or the far cross walk during the green phase, triggering a no right-turn-on-red or cautionary pedestrian crossing, respectively, by utilizing a blank signal sign.

According to an aspect of the invention, a method for controlling a traffic signal directed to vehicles travelling on a roadway to which the traffic signal is directed includes automatically monitoring an area adjacent a pedestrian crossing so as to detect the presence of a pedestrian therein, and detecting physical parameters of the pedestrian. From the physical parameters of the pedestrian, it is determined whether there is a likelihood that the pedestrian will cross the pedestrian crossing. Responsive to a determination that the pedestrian is likely to cross the pedestrian crossing, causing the traffic signal to display a directive to the vehicles so that the vehicles permit the pedestrian to cross the pedestrian crossing.

According to another aspect of the invention, an electronic system automatically controlling a traffic signal directed to vehicles travelling on a roadway to which the traffic signal is directed with a pedestrian crossing across said roadway includes a controller controlling the traffic signal so as to selectively display thereon directives to at least some of the vehicles on the roadway. An automatic pedestrian crossing system is connected with the controller. The automatic pedestrian crossing system has at least one camera having a field of view covering an area at least adjacent to the pedestrian crossing. A computerized pedestrian monitoring system is connected with the camera and receives therefrom an electronic video signal comprising a series of frames of the field of view of the camera. The pedestrian crossing system determines from the electronic video signal if a pedestrian is present in the area adjacent the pedestrian crossing, and responsive to a determination that a pedestrian is present in the area for a period of time of at least three seconds, derives pose estimation data from the video signal corresponding to a pose of the pedestrian in the area. The pedestrian crossing system has a neural network trained to differentiate between poses of pedestrians that intend to cross the pedestrian crossing and pedestrians in the area that do not have the intent to cross the pedestrian crossing. The neural network processes the pose estimation data and derives from it a determination whether or not the pedestrian has an intent to cross the pedestrian crossing. The pedestrian crossing system is connected with the controller and transmits to the controller an electronic command responsive to a determination that the pedestrian has an intent to cross the pedestrian crossing. Responsive to receiving the electronic command, the controller is configured to cause the traffic signal to enter an active pedestrian crossing state wherein the traffic signal displays a traffic directive configured to avoid conflict of some of the vehicles with pedestrians in the pedestrian crossing.

According to another aspect of the invention, a reliable system is provided that predicts pedestrian crossing intentions, proactively activating the pedestrian phase if they forget to press the button before crossing, or pressing both buttons or the wrong button, to ensure their safe crossing. The implementation of a real-time system to estimate the crossing intention of the pedestrians at an intersection uses video feed from four cameras strategically placed at four-legged intersections. Each camera points to one of the four waiting areas. Real-time processing has been achieved by efficiently integrating Yolov8 and OC-SORT for pedestrian detection, tracking, and pose estimation. The pose estimation will be only active when the pedestrian is in the waiting area to reduce the computational demand and decrease the latency.

According to still another aspect of the invention, a method for controlling a traffic signal directed to vehicles travelling on a roadway to which the traffic signal is directed comprises electronically sensing a presence of a pedestrian in an area of a pedestrian crossing and automatically in real time making a determination based thereon of an intention of the pedestrian to cross the pedestrian crossing. Responsive to the determination of the intention of the pedestrian to cross the pedestrian crossing, the traffic signal is caused to display a directive to vehicles on the roadway so as to permit the pedestrian to cross at the pedestrian crossing.

The sensing and making of the determination of the pedestrian crossing is performed using a camera generating an electronic signal that comprises video of the pedestrian in said area. The making of the determination includes deriving location data and pose data for the pedestrian and making the determination of the intent of the pedestrian to cross the pedestrian crossing from it. The location data includes data identifying the pedestrian crossing and data indicative of a distance of the detected pedestrian from the pedestrian crossing, and the pose data comprises data indicative of position of a head or upper body of the pedestrian. The causing of the traffic signal to display the directive is either performed by direct control of the traffic signal or by transmitting an electronic signal to a controller that is configured to control the traffic signal responsive to activation of a pedestrian push button, and the electronic signal sent to the controller is in that case treated by the controller as a signal from the activation of the pedestrian push button when pushed.

In the standard traffic signal controller, as is well known in the art, the controller receives the electrical signal from the pedestrian call button, and begins to process service of the pedestrian call which usually involves the controller cycling through the programmed phasing sequence for the intersection until it reaches an appropriate time in the cycle to implement the pedestrian crossing phase signal pattern, which usually involves displaying a red signal for the vehicular phase that would conflict with the pedestrian crosswalk, and display of a WALK signal or the walking man symbology to display to the pedestrian. The cycle may be a local cycle or part of a cycle received from a traffic control system that implements a traffic control pattern for the intersection in combination with other intersections in the area. The pedestrian crossing signal pattern is displayed for a predetermined period of time determined by the traffic engineers as sufficient to allow most pedestrians to cross the crosswalk involved. The pedestrian crossing signal pattern then cycles back to the pre-existing vehicle pattern without the pedestrian call signal.

The system of the invention can function in this way, i.e., by generating a typical pedestrian call function as above based on detection of a pedestrian intent to cross. The preferred embodiment preferably adds other functionalities to the more conventional pedestrian call function, as will be described herein.

The making of the determination of the intent to cross is by computer system that is an AI system, or a computer provided with programming derived from an AI system. The AI system is trained to differentiate between pedestrians that cross at a crosswalk and pedestrians that are sensed but do not cross by providing to the AI system:
a number of historical pedestrian location and pose data sets; and
for each of the pedestrian pose data sets a respective historical crossing result data indicative of whether the pedestrian crossed the crosswalk.

The directive may be a red light or a no-turn indicator of a traffic light of the traffic signal.

The method of may further comprise sensing the pedestrian in the crossing, and determining whether the pedestrian is still in the pedestrian crossing after elapse of a time period from the display of the directive. Responsive to a determination that the pedestrian is still in the crossing, the method provides for extending a period of time, based on the walking speed of the pedestrian, for the display of the directive for the traffic signal long enough to allow the pedestrian to complete crossing of the pedestrian crossing.

Where the method is applied to an intersection having two or more phases of traffic control, the method may include sensing other pedestrians at least one area adjacent at least one other pedestrian crossings for which at least one other traffic signal is configured to display directives to vehicles affecting said at least one other pedestrian crossing, and determining whether pedestrians in the area have an intent to cross the other pedestrian crossing, and responsive to a determination of the intent, causing the other traffic signal to display a directive against vehicles entering the pedestrian crossing in the pedestrian crossing associated therewith.

The making of the determination is by a computer system that is either a trained neural network or other AI system, or a computer provided with programming derived from training an AI system. The training of the AI system teaches it to differentiate between pedestrians that cross at a crosswalk and which crosswalk the pedestrian is intending to cross when two or more different crosswalks are in the field of view of the associated camera, and pedestrians that are sensed but do not cross. This is done by providing to the AI system:
a number of pedestrian pose data sets; and
for each of the pedestrian pose data sets a respective crossing result data indicative of whether the pedestrian crossed the crosswalk, and identifying which crosswalk was crossed, if any.

The present system addresses the shortcomings of the prior art by offering safe and efficient crossing experiences for pedestrians and reducing unnecessary vehicle delays during the pedestrian phase. This approach not only improves pedestrian safety and experience but also enhances overall traffic fluidity and reduces congestion.

The present system integrates real-time processing and specifies crosswalk intentions, thereby offering a more sophisticated and effective solution for pedestrian safety at intersections. The system provides a comprehensive solution utilizing advanced computer vision and deep learning techniques to anonymously identify pedestrians' pose estimation and movements in real-time, to predict crossing intentions, and to proactively activate appropriate phase signals. The system also, through real-time monitoring, enables comprehensive tracking of pedestrian movements at crosswalks, allowing for the proactive extension of crossing duration for disabled or slow-moving road users. This also ensures that conflicting vehicle phases are not activated when a pedestrian is present on the crosswalk.

By integrating these features, the system can substantially reduce pedestrian-vehicle collision risks, foster a safer, more sustainable transportation environment, and improve the efficiency of the intersection.

The system offers several objects and advantages:
Enhanced Safety for Vulnerable Road Users: The system predicts the intentions of pedestrians and other vulnerable road users, such as cyclists, scooter riders, and powered wheel riders, with high accuracy in real-time. This enables proactive measures to ensure their safety and reduce the risk of accidents.
Active Monitoring: The system's phase extension checker actively monitors pedestrians, enabling the extension of crossing phases to ensure their safety. This feature significantly reduces the risk of accidents and near-misses, providing a safer environment for all road users.
Reliable Performance in Adverse Conditions: the system performs well even in challenging weather conditions, such as light rain, and during night-time hours. This reliability ensures that the system remains effective in a wide range of scenarios.
Efficient Intersection Management: The system serves all road users at intersections by improving pedestrian safety and signal performance. It can predict the crossing direction of pedestrians, proactively activate the phase, and reduce crossing violations. Additionally, it minimizes false calls of the pedestrian signal, making the signal more efficient for all road users.
Easy Implementation and Scalability: The system is easy to implement and can be scaled to many other locations and states, making it a versatile and effective solution for enhancing pedestrian safety. Its adaptability and ease of implementation make it an attractive solution for cities seeking to improve pedestrian safety and reduce accidents.
Cost-Effective Solution: the system offers a cost-efficient implementation, and through Artificial Intelligence it can achieve significant results without requiring major infrastructure changes at intersections. By utilizing regular cameras to perceive the environment, the system can run its advanced capabilities, making it a budget-friendly solution for cities seeking to enhance pedestrian safety.

The system is a highly scalable and versatile solution, capable of seamlessly integrating with various road junctions, including intersections and midblock crosswalks, to enhance pedestrian safety. The system is designed such that it can be easily integrated to existing signal controllers across various vendors. The cameras used for this work also use existing traffic mast arms, thereby avoiding additional costs such as installing new poles. As such, it can be widely adopted across state-wide and country-wide intersections with minimum cost.

Leveraging the power of Artificial Intelligence and real-time processing, this system boasts high monitoring capabilities, making it an ideal deployment-ready solution for saving lives. By harnessing cost-efficient tools, the system proactively predicts pedestrian crossing intentions, triggering the corresponding pedestrian phase and mitigating jaywalking. Moreover, its robust design enables it to cater to a diverse range of vulnerable road users, including pedestrians, cyclists, and powered wheel riders, across various weather and lighting conditions. This intelligent system can be easily integrated into any intersection, optimizing signal control logic to strike a perfect balance between safety and efficiency.

The system utilizes pedestrian surveillance cameras and deep learning to predict pedestrian crossing intentions at specific intersections, achieving a 94.67% accuracy rate. This also eliminates false calls, which optimizes signal performance. It is anticipated that this system can reduce pedestrian fatalities and injuries by half within a few years.

Other objects and advantages of the invention will become apparent from the present specification.

### BRIEF DESCRIPTION OF THE DRAWINGS

FIG. 1A is a diagram of the general configuration of the components of a pedestrian crossing installation according to the invention.

FIGS. 1B to 1F are diagrams showing the placement of cameras at intersections and their coverage areas in which there is image capture of pedestrians and other VRUs.

FIG. 2 shows a data collection setup with a camera at an intersection.

FIG. 3 is a diagram that illustrates the overall system operation and hierarchy.

FIG. 4 is a diagram that shows the zones of interest and the pose components employed in the system.

FIG. 5 is a set of views showing the zones of interest for each camera view and relevant points on VRUs for determining crossing intention.

FIG. 6 shows views of an intersection with pedestrian phase extension functionalities.

FIG. 7 also shows views of an intersection with pedestrian phase extension functionalities.

FIG. 8 shows graphs of feature importance relative to different components of a model used in the system.

FIG. 9 is an illustration of distortion of keypoints of human body leg and arm joints due to shadow in visual processing of the pedestrian's pose.

FIG. 10 shows camera imagery for some crossing intention predictions in daytime.

FIG. 11 shows camera imagery for some crossing intention predictions at night.

FIG. 12 is a diagram showing types of VRUs in the test dataset.

FIG. 13 shows images associated with activation of the pedestrian extension checker in different situations.

FIG. 14 is a flowchart of the pedestrian monitoring system operation for detecting and using pedestrian crossing intent.

FIG. 15 shows scenarios in which it is advantageous to provide specific traffic signal messages to vehicles to prevent conflict with detected pedestrians.

FIG. 16 illustrates the internal steps of the computerized system in the processing of the scenarios of FIG. 15.

### DETAILED DESCRIPTION

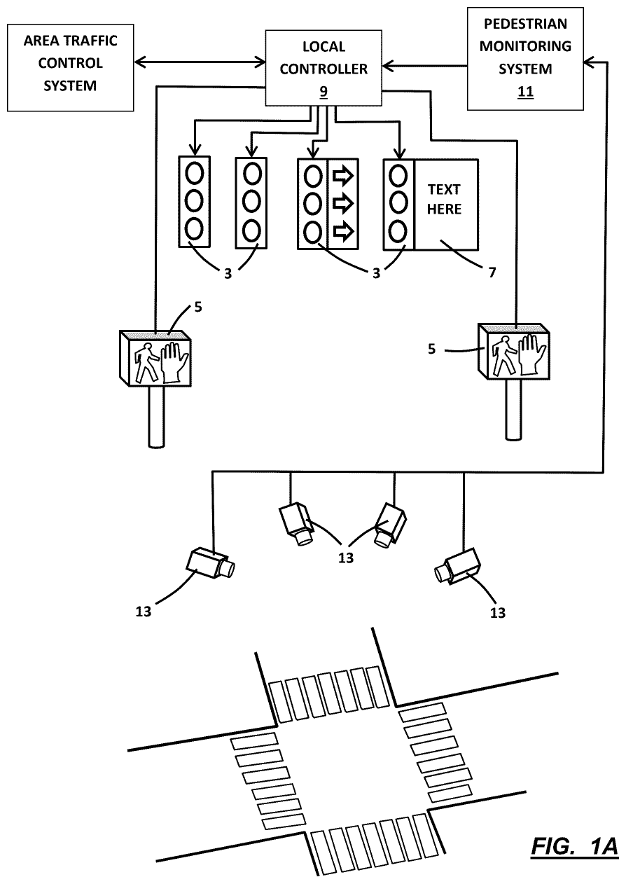

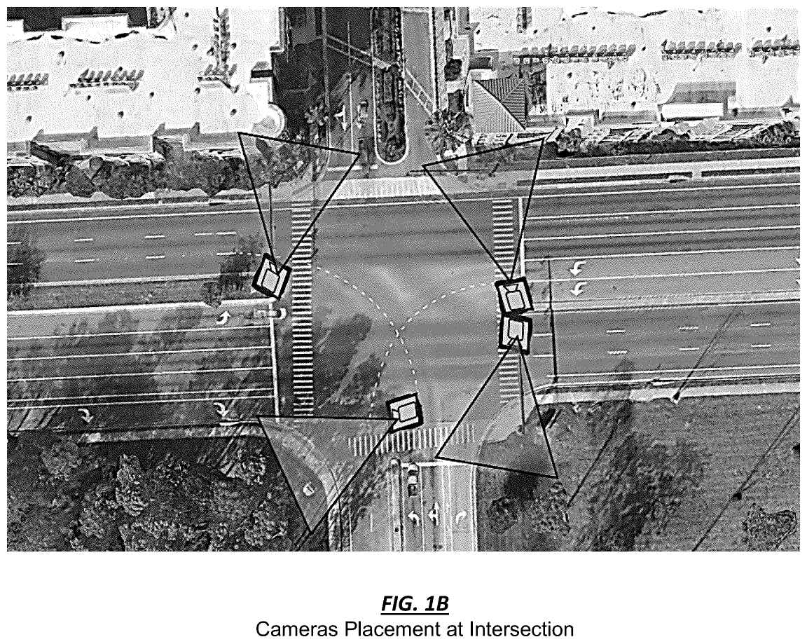

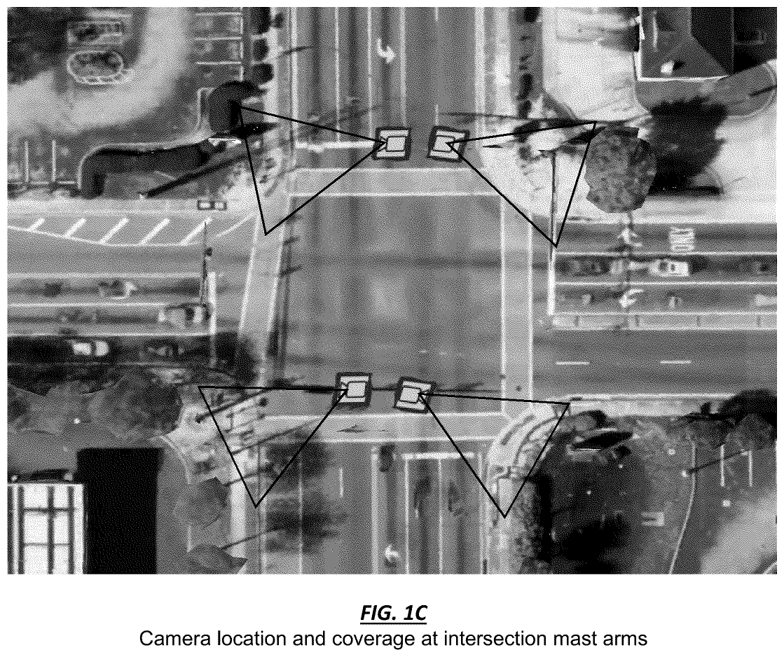

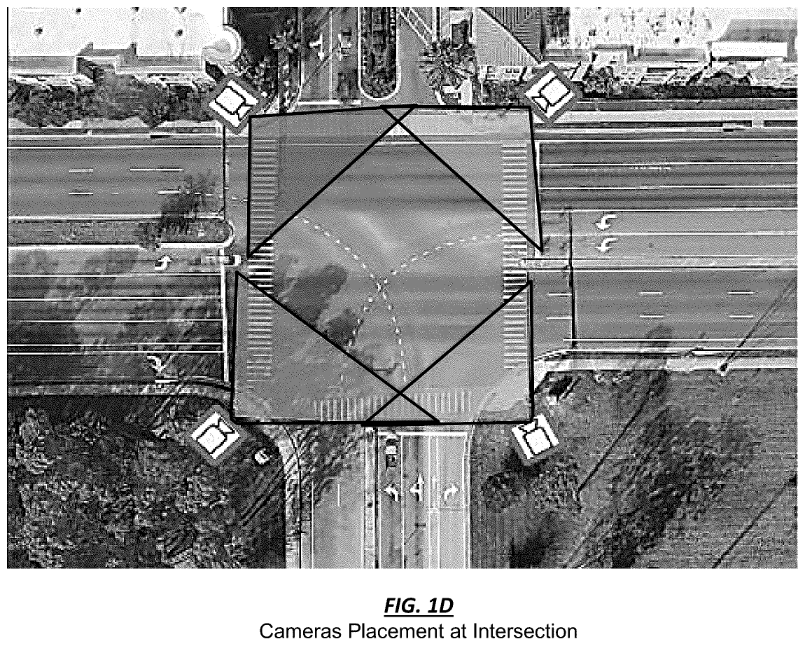

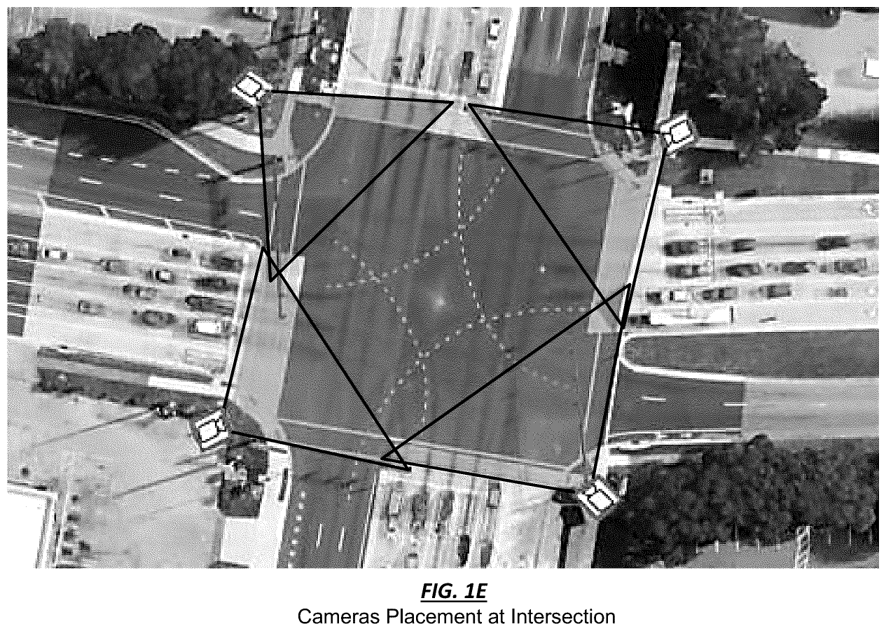

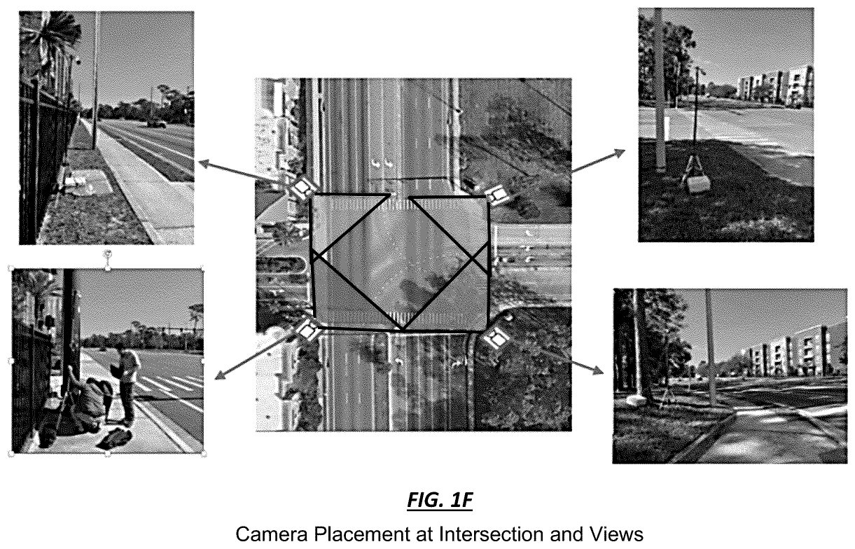

As illustrated in FIG. 1, the system of the present invention is implemented in the context of a pedestrian crossing or crosswalk across a roadway. The location can be as simple as one crosswalk and one lane of traffic, or it may have multiple lanes of vehicular traffic with several crosswalks across the various lanes.

Whichever sort of crossing environment is involved, the system includes one or more vehicular traffic control signal devices 3, and at least one crosswalk, preferably with at least one pedestrian crossing signal 5. The vehicular traffic control signal devices 3 display the usual well-known traffic control indications or directives, such as red lights, amber lights, green lights, or red, green, or yellow arrows, or any other signals that modify vehicular traffic movement. The signal devices 3 may also include devices 7 that display more complex traffic commands, such as written or pictorial displays that direct vehicles to take or not take some action, such as, e.g., NO TURN ON RED. The pedestrian signals 5 indicate to pedestrians when it is safe to cross a corresponding crosswalk and when they should wait. Commonly the pedestrian signals 5 selectively display the words WALK or DON'T WALK or nonverbal images with similar meanings, and sometimes numerical countdowns to the end of the pedestrian crossing interval.

As is well known in the art, the traffic and pedestrian signals 3, 5, and 7 are electronically controlled locally by a controller 9 situated at the location of the traffic signal or signals. The controller controls all of the traffic signals 3, 7 and pedestrian indicators 5 at the associated location or intersection.

The controller 9 may operate independently or semi-independently based on local conditions, based on local vehicle detectors, and/or based on pedestrian buttons, if present. The operation of the controller is also normally controlled by direct electronic commands from a municipal or other area computerized traffic control system located remotely the controller to implement a traffic control pattern with other traffic lights at other intersections in the area.

The system includes an additional computerized pedestrian monitoring module 11 connected with one or more cameras 13 of a sort well known in the art of traffic monitoring or general surveillance functions. The cameras are directed to one or more areas associated with the pedestrian crossings, preferably areas on the sidewalk adjacent the crosswalk where pedestrians stand before they cross the street, and also at the crosswalk in the roadway so the system can monitor pedestrians as they are actually crossing.

As will be described below, the computerized pedestrian monitoring system 11 automatically detects from the video signals the presence of pedestrians in areas adjacent the crossings in real time, and determines from the video if the detected pedestrian has an intention to cross at one of the crossings. When the intention to cross is determined, the system automatically causes the traffic signals and pedestrian signals to stop or alter traffic away from the associated crosswalk, and to display the WALK pedestrian signal.

In one embodiment, the system of the pedestrian monitoring system 11 is separate from the controller 9. When a pedestrian intent to cross is determined by system 11, it effectuates pedestrian crossing by transmitting a signal to an input of the controller that is used in prior art systems to receive an electrical signal from a pedestrian push button that a pedestrian pushes to cross the street and request a pedestrian walk phase of the traffic lights. In other words, the system 11 emulates a pedestrian pressing the crossing button at the intersection without need of the pedestrian to actually push the button.

Alternatively, the pedestrian monitoring system 11 may be integrated into the controller 9, and, in that case, the electronics of the controller include a processor and associated computer memory and software that both controls the traffic lights and also provides the pedestrian intent monitoring and other electronic functionality of the present invention completely internally to the controller.

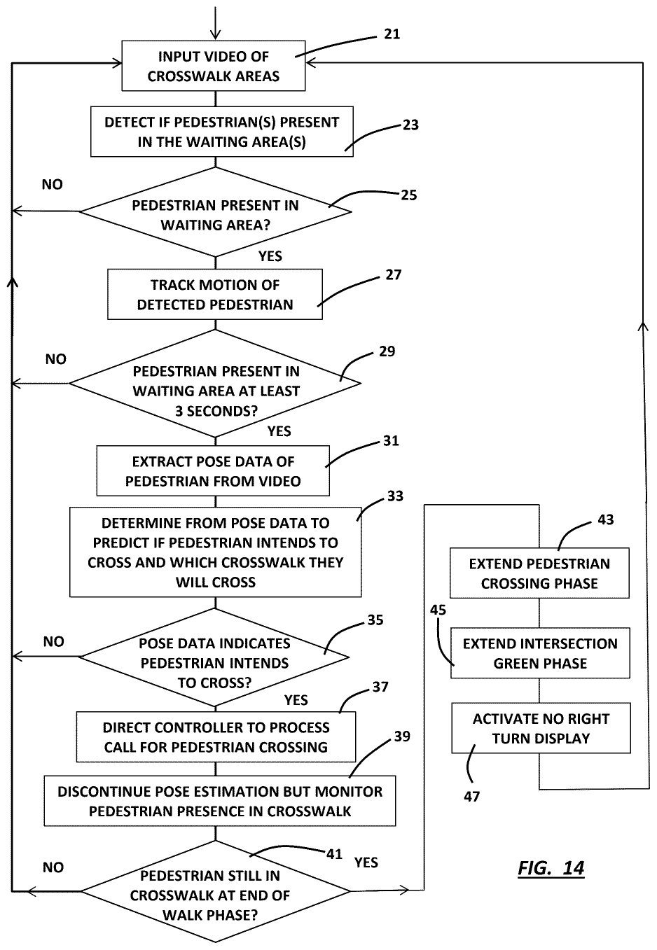

The flowchart of FIG. 14 illustrates operation of the system.

The computer system 11 continuously receives the video from the cameras at the intersection in step 21. It then processes the video stream using computer vision methods (step 23) to detect if any pedestrian is present in any of the areas adjacent or in the crosswalks that are being surveilled. When a pedestrian is detected (step 25), the system tracks their movements (step 27). If motion analysis indicates that the pedestrian is likely to cross, evidenced by remaining nearly stationary for 3 seconds in the waiting area (step 29), pose estimation data will be extracted along with other features (step 31) and sent to the machine learning model to predict the crossing intention of the detected pedestrian (step 33). If the pedestrian does not remain stationary, it is determined that the pedestrian is not intending to cross and is just passing by. When the system determines that the pedestrian intends to cross at a cross walk (step 35), the system generates a signal directing the controller of the intersection of a pedestrian crossing call for the crosswalk for which the intent to cross is identified (step 37), similar to the signal produced by pushing of the pedestrian crossing button in earlier systems, as will be described below.

Once the pedestrian begins crossing the crosswalk, pose estimation is deactivated, and only detection and tracking remains active to monitor the pedestrian's movement while crossing (step 39). The pose estimation model is only required when predicting the crossing direction, which is necessary when the pedestrian is in the waiting area; otherwise, pose estimation is not needed and is not performed.

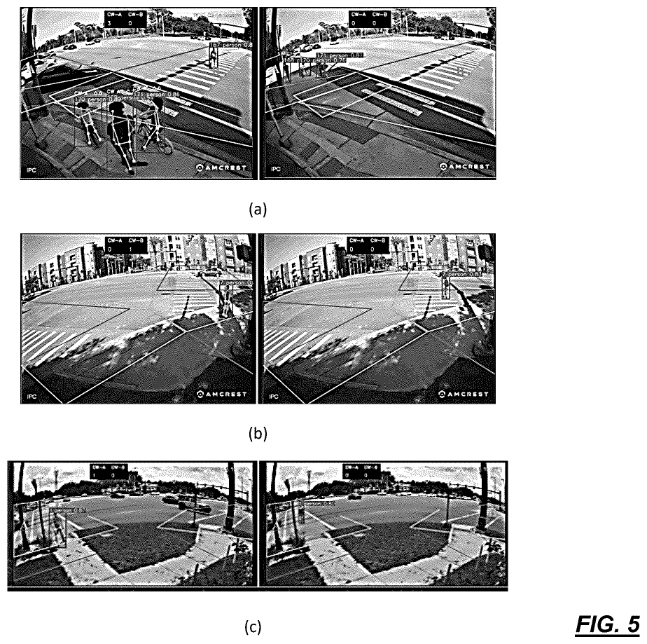

The location data for the pedestrian includes data that identifies which crosswalk the pedestrian is in or adjacent to, and the pedestrian's relative location and distance from it. This data is derived by a process that involves identifying the pedestrian's location and calculating from that location the relative distance of the pedestrian to the start of the crosswalk, or to the start of each of the two or more crosswalks that may be present in the video image stream. Additionally, a start-crossing zone is defined for each crosswalk, and the location of the pedestrian is used to determine if the pedestrian is in the start-crossing zone of the associated crosswalk. The determination of the pedestrian being in the start-crossing zone of one of the crosswalks is used as a strong indication that the pedestrian is in close proximity to either of the crosswalks. This factor is important in some layouts where the waiting area of the crosswalks are separated as seen in FIG.5(b and c). However, if the crosswalks are so near to each other this factor is not reliable as in FIG.5(a), and pose data is so important and is used to predict the crossing direction.

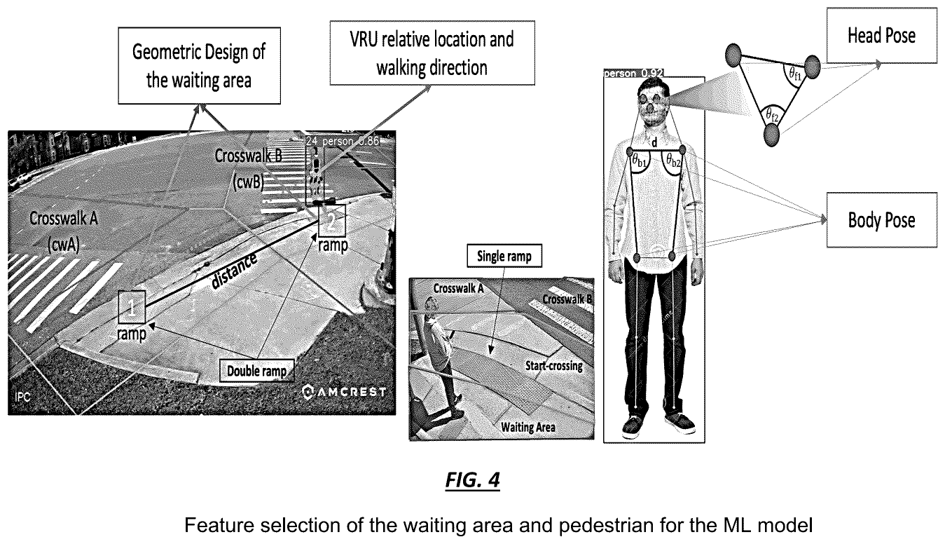

The pose data indicates the orientation of the body and the head of the pedestrian, i.e., the direction that the pedestrian is facing and/or looking. The pose data includes the pixel coordinates of 17 key points on the human body, such as the head, shoulders, elbows, and knees. These key points, shown in FIG. 4, mark important spots on the body to help understand the person's position and movement in the image. The pose estimation model outputs the 17 keypoints of the human body as pixel locations (x,y) in the image. Table 1 below exemplifies the pose data.

The location and pose data along with data that defines the geometric design of the waiting area (including the number of ramps and distance between the start of the crosswalks) are then processed by the AI system 11. The AI system has been trained to process the pedestrian video signals from the cameras and determine whether or not there is a crossing intent of the pedestrian and issues a determination or flag that the pedestrian intends to cross the crosswalk, or that the pedestrian does not intend to cross.

Referring again to FIG. 14, if there is no determination of intent to cross, then the system loops back and repeats the process for the next video frame being received from the cameras.

On the other hand, if there is a determination of an intent to cross (step 35), the system 11 outputs a signal to the controller 9 that causes the controller to change the intersection traffic lights to a pedestrian crossing phase for the crosswalk for which the pedestrian intent to cross was determined (step 35). According to one embodiment, this is accomplished by transmitting a signal to the controller that emulates a pushbutton pedestrian cross request. In response, the controller transitions the state of the traffic lights and signals to one that corresponds to a pedestrian crossing phase for the identified crosswalk.

The pedestrian crossing phase normally has a predetermined duration of a period of time sufficient for the pedestrian to completely cross and clear the intersection before vehicular traffic is permitted by the traffic signals.

Preferably, the system monitors the video signals during the period of time of the pedestrian phase (step 39), and determines if the pedestrian is still in the crosswalk as the period of time is ending (step 41). If the pedestrian is still present in the crosswalk as the period of time is expiring, the system 11 preferably directs the controller 9 to extend the pedestrian crossing phase by an additional period of time to permit the pedestrian to finish crossing the crosswalk before returning the signals to the vehicular flow across the crosswalk (step 43).

The signal controller is configured such that it can cancel a pedestrian call responsive to an input or determination that it is not needed. Where a pedestrian is determined to have an intent to cross the crosswalk by the system, but the subsequent video shows the system that the pedestrian has, in fact, not crossed, but has left the crosswalk and the crosswalk starting area, the system then directs the signal controller to cancel or shorten the pedestrian crossing phase pattern for that crosswalk.

The signal controller also can extend the green phase of vehicles moving through the intersection in parallel with the crosswalk being used by the pedestrian, so as to protect pedestrians that did not complete the crossing in time (step 45). For such an installation, a blank sign is also added to the traffic signals and is controlled (step 47) to display a traffic directive (such as NO RIGHT TURN) to address potential pedestrian conflicts with right turning vehicles.

### Dual Blank Sign for Warning Vehicles and Ensuring Safe Crossing

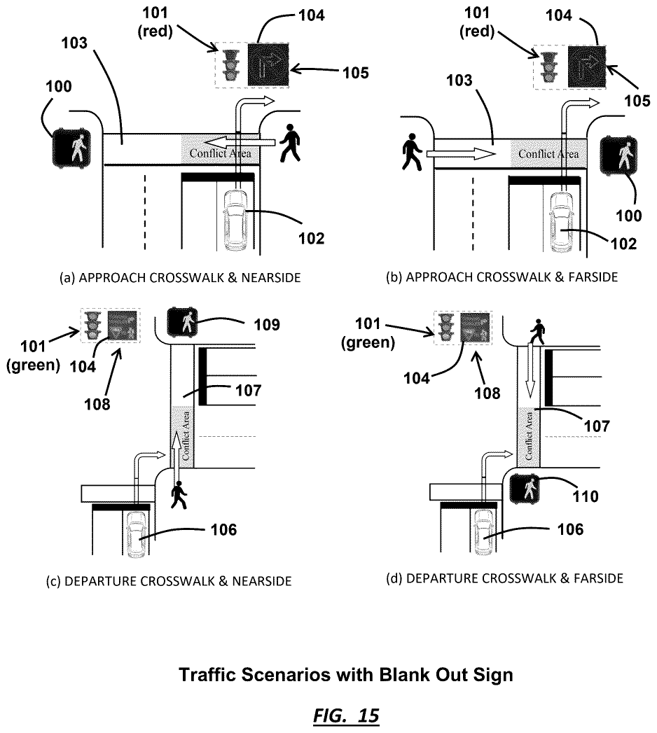

The operation of the “blank sign”, i.e., a sign that can display a detailed text or image traffic directive to vehicles, is shown in FIG. 15.

Driver-related crashes are primarily caused by behavioral violations such as failure to yield to pedestrians, careless driving, driver misconduct (e.g., impaired driving), and driver qualification issues like unlicensed or inexperienced drivers. Among these, failure to yield and careless driving are the most significant contributors, leading to a large proportion of severe injuries. On the pedestrian side, risky behaviors such as failure to obey traffic signs or signals, failure to yield right-of-way, and improper roadway behavior (e.g., walking outside designated areas) are major contributors to crash risks. These behaviors often introduce unpredictability, leaving drivers with minimal reaction time, particularly under conditions of low visibility or when driver reaction times are delayed.

This complexity in driver and pedestrian behaviors is further reflected in Surrogate Safety Measures such as Post Encroachment Time (PET) and Relative Time-to-Collision (RTTC), which provide insights into the risk levels associated with different crosswalk locations.

At an intersection, the approach crosswalk is the one encountered first by vehicles entering the intersection. In contrast, the departure crosswalk is the one through which vehicles exit the intersection area.

Both the PET and RTTC median values for the departure crosswalk are smaller than those for the approach crosswalk, indicating that departure crosswalks pose a higher risk for Right Turn conflicts compared to approach crosswalks. This could be affected by pedestrian-related factors such as driver reaction time or restricted visibility caused by in-vehicle or external obstructions that limit the driver's ability to fully see pedestrians about to cross, which can vary greatly during right-turn maneuvers. To address these challenges, infrastructure improvements like installing warning signs, are essential.

As best shown in FIG. 15, a dual blankout sign is installed next to the red-yellow-green traffic signal to warn drivers. Showing a warning message of a “NO TURN ON RED” sign prohibits a right turn (during red) that can conflict with a prior pedestrian crossing phase, and ensures safe crossing. Another warning sign, “TURNING VEHICLES YIELD TO PEDESTRIANS”, warns drivers to proceed cautiously (during green) and prioritize pedestrians in crosswalks.

As best seen in FIGS. 15(a) and 15(b), when the red light 101 is illuminated, vehicles such as vehicle 102 stop for the red light 101. However, under Right Turn on Red (RTOR) traffic regulations, vehicles are permitted to turn right despite the red light. At the same time, the pedestrian crossing phase signal 100 usually will indicate a WALK signal to pedestrians crossing the lane of cars 102 stopped for the red light 101. This can create potential conflict between the right of way for pedestrians and vehicles turning right on red, i.e., with pedestrians crossing the approach crosswalk 103 either from the nearside of the crosswalk (FIG. 15(a)) or the farside of the crosswalk (FIG. 15(b)).

To address this conflict and enhance pedestrian safety, according to the present invention, when the presence of a farside or nearside VRU in the approach crosswalk 103 is detected by the computer vision system or predicted based on the detected intent of a VRU to cross by the pedestrian detection system, the blank out sign 104 is activated to display a “NO TURN ON RED” message 105.

Unlike static signs, in the preferred embodiment, the blank out sign is preferably activated only during pedestrian crossing phase where indicated appropriate by the computer vision algorithms, meaning that the “NO TURN ON RED” blank out sign display is implemented only when a VRU or pedestrian is detected. At other times, the blank out signs are cleared or blank. This ensures pedestrian safety without added intersection delay since it is only activated when pedestrians are detected.

As best shown in FIGS. 15(c) and 15(d), another potential conflict arises during right-turn on green. In these situations, the approaching vehicle 106 reaches the intersection with the traffic light 101 showing a green light, and proceeds to turn right. At the same time, there may be a pedestrian crossing the departure crosswalk 107 in parallel with the green-light lanes with a WALK indicator 109 or 110, and in the path of the right-turning vehicle. This results in a conflict between pedestrians crossing the crosswalk and vehicles making a right turn.

The system of the preferred embodiment mitigates this conflict by causing the blank-out sign 104 to display a “TURNING VEHICLES YIELD” to pedestrians message display 108, alerting drivers to yield to pedestrians, reinforcing the need for caution and prioritizing pedestrian right of way. This display may may be displayed only on detection of a VRU or pedestrian in the departure crosswalk 107, or it be on constantly during the green-light phase of traffic signal 101.

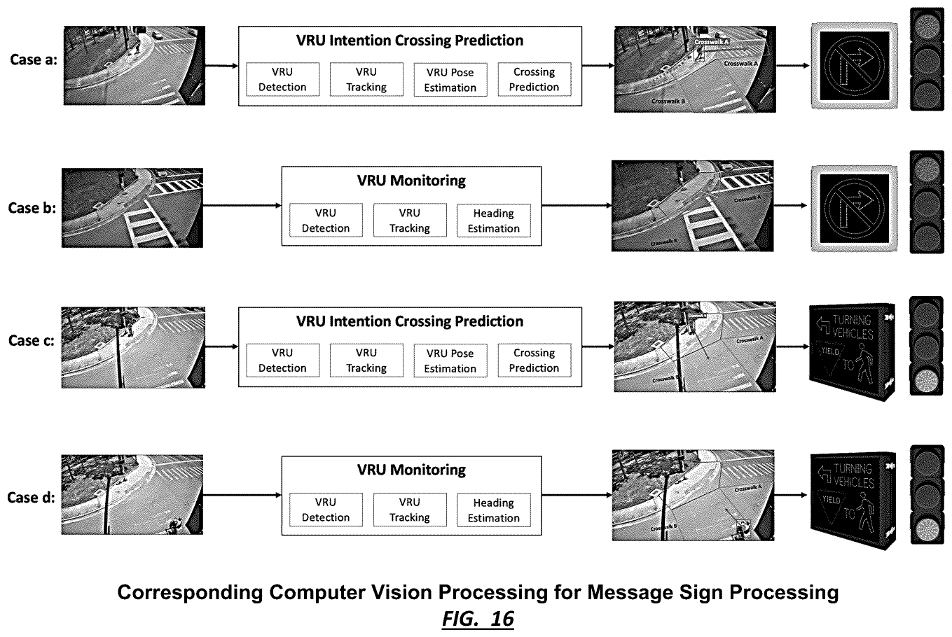

FIG. 16 illustrates the corresponding computer vision processing for the four different cases presented in FIG. 15, and described above. In the scenarios of FIG. 15(a) and FIG. 15(c), when the system predicts a crossing, it uses the full process to analyze VRU behavior and predict the crossing direction, and then implements the blank out sign display for either “NO TURN ON RED” or “TURNING VEHICLES YIELD”. However, if the VRU is crossing from the far side, as in the scenarios of FIG. 15(b) and FIG. 15(d), the system only applies detection and tracking without running the full process. This helps the system work efficiently while still keeping track of VRUs.

### Determination of Intent or Lack of Intent to Cross

The determination of the intent (or lack of intent) of the pedestrian to cross at the crosswalk is made based on the location data and pose data for the pedestrian when detected by an AI system 11. The AI system is trained to recognize the pedestrian intent to cross by training the system with a set of training data sets. Each of the training data sets includes
a. a number of input data sets each comprising
   i. pose data defining a position of the head or eyes or the body, or all of these, of a respective pedestrian in an area adjacent a crosswalk; and
   ii. location data of the pedestrian at the area adjacent the cross walk; and
b. for each of the input data sets, crossing result output data indicative of whether the pedestrian with the pose and location defined did, in fact, cross the street at the crosswalk, e.g., usually as a yes or no, 1 or 0, value.

In one embodiment, the AI system was trained on two intersections involving 437 VRUs including pedestrians, cyclists, and e-scooter riders, all captured during daytime only. The trained AI system was then tested on a completely different third intersection with 589 VRUs involving more other VRU types than in the training dataset such as e-bike riders, e-skateboard riders, and kids, during both daytime and nighttime conditions.

The system here described, herein referred to as VRU-CrossSafe or Ped-CrossSafe, is a real-time pedestrian monitoring and crossing prediction system that harnesses the power of advanced sensing technology and artificial intelligence to enhance road safety. This comprehensive system has demonstrated outstanding results in predicting crossing intentions for pedestrians during both daytime and nighttime, achieving real-time processing with an average accuracy of 94.67% and average video processing speed of 33 FPS.

By facilitating the automatic activation of pedestrian signals, VRU-CrossSafe eliminates the need for push-buttons and reduces false calls, thereby enhancing signal performance and promoting a safer and more efficient traffic flow. Additionally, the inclusion of a phase extension checker enables the system to monitor pedestrians on the crosswalk, ensuring sufficient time is allocated for them to cross safely and comfortably.

According to the preferred embodiment, video feeds from at least four strategically positioned cameras at four-legged intersections, such as shown in FIGS. 1B to 1E, are used by the system utilizing advanced deep learning and computer vision techniques to detect, track, and estimate pedestrian poses with exceptional accuracy, including vulnerable populations such as toddlers, disabled individuals, cyclists, and powered wheel riders. The fields of view of the cameras preferably include each of the waiting areas for pedestrians, and also at least portions of the associated crosswalks. The actual placement of the cameras may vary, and they may be on the mast arm of the roadway (as in FIG. 1B) or looking inward from four corners (as in FIGS. 1C, 1D, 1E, and 1F).

The system is automatic and computerized, and the cameras transmit electronic signals, usually digital video, that are transmitted to a computer that has a processor and associated computer-accessible memory, and supports the operation of the system as described herein. The computer is connected electronically to the traffic signals, usually via a wireless or hardwire connection through the controller located at the intersection that controls the visible traffic signals for the intersection.

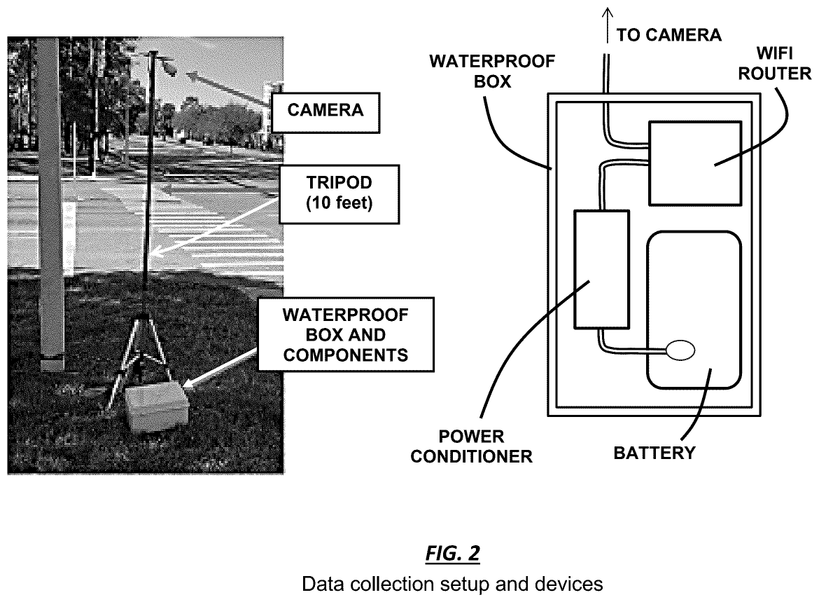

Individual camera units may be functional units as shown in FIG. 2, where the camera is supported on a 10 foot tripod, and is connected to a waterproof container for the power and communications components of the camera unit, usually a battery connected to a power conditioner powering the camera itself and aa Wi-Fi router that transmits video to the computer system 11 at the intersection.

### Models for Processing Video

The system preferably employs YOLOv8 and OC-SORT, which are deep learning models recognized for their proficiency in VRU detection, tracking, and pose estimation. The primary objective is to predict VRU crossing intentions in real time at intersections, thereby ensuring safe crossing. Additionally, the utilization of these models allows for improved traffic signal performance, particularly in situations where false calls may occur due to pedestrians pressing the wrong push button or multiple buttons simultaneously.

At a typical four-leg intersection, there are four waiting areas where each waiting area serves two crosswalks. Pedestrians, cyclists, e-scooter riders, and others at intersections are vulnerable to vehicle incursions during the walk phase, which can lead to severe injuries or fatalities. Moreover, research shows that many VRUs do not push buttons to activate the targeted pedestrian phase, and those that do so often push both buttons if the button placement is unclear. Therefore, monitoring VRU movements is helpful for predicting crossing intentions in real time to ensure safety while crossing and improving signal performance by removing false calls.

### System Pipeline Hierarchy

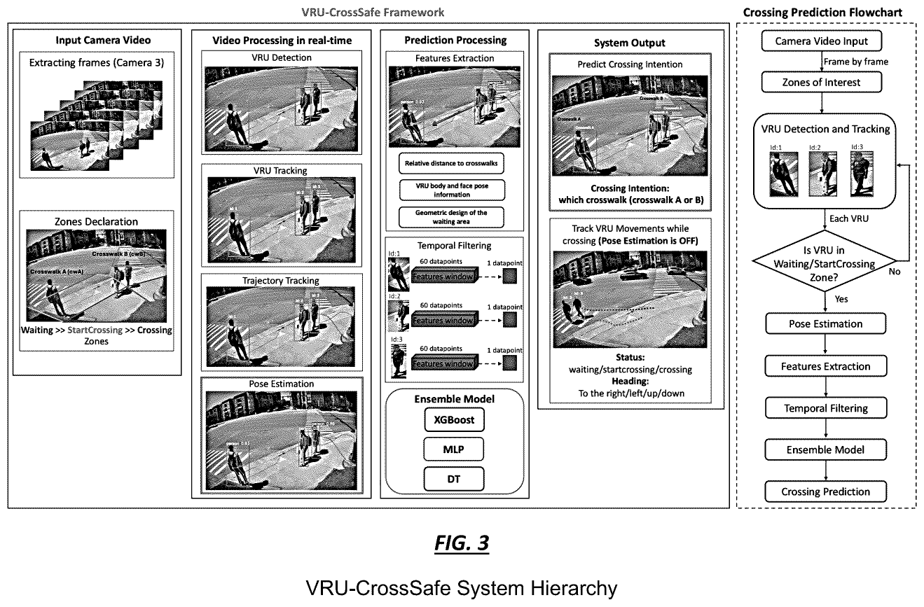

FIG. 3 illustrates the method hierarchy employed for pedestrian crossing intention prediction, which has four main stages performed by the computer of the system.

First, frames are extracted from the streamed video on a frame-by-frame basis, and the zones of interest are declared. These regions are the waiting, start-crossing, and crossing zones. A start-crossing zone is added because some pedestrians start approaching the crosswalk while the pedestrian crossing phase is not yet activated, but the pedestrian in this zone has a high potential for crossing regardless of the current status of the traffic signal. Through trajectory tracking of pedestrians, their location within the declared zones can be determined.

In the second stage, these frames are processed utilizing computer vision and deep learning capabilities. The movements and actions of pedestrians are monitored through detecting, tracking, and estimating their pose. YOLOv8 is a deep learning model for pedestrian detection and estimating keypoints in which two models are used, one for VRU detection and the other for pose estimation, and both are in the “medium” version, where YOLOv8 offers a range of five models, varying from the fastest with the lowest performance to the slowest with the highest performance.

The detection model takes an image, which is a video frame in this system, and scans it to detect the objects of interest in the image, providing accurate bounding boxes for each detected object. These bounding boxes represent the object's location by defining a rectangular area around it, typically characterized by data defining the coordinates of its top-left corner, width, and height. In addition, the detection model generates data that identifies the class of each detected object, such as whether it belongs to the person class, car class, dog class, etc.

OC-SORT is an algorithm for tracking pedestrians through frames to extract the corresponding trajectories and keep the movement records of each pedestrian. The algorithm produces data that includes unique IDs for each pedestrian, bounding box coordinates in each frame imported from the detection model, and detailed trajectory information that tracks pedestrians' movement over time. It also records the position and velocity of pedestrians, allowing for accurate motion tracking even when occlusions occur. The key output from OC-SORT is the continuous trajectory data, which maps each VRU's movement through successive frames, making it valuable for analyzing pedestrian movements.

Through trajectory tracking, the walking speed and heading are calculated, and based on the pose estimation, the angles and distances between body joints are calculated. These features are needed for the crossing prediction machine learning model for pedestrian crossing intention prediction.

The third stage involves feeding the extracted information for each pedestrian to a computer-implemented ensemble model that integrates three distinct types of models, namely, eXtreme Gradient Boosting (XGBoost), multilayer perceptron (MLP), and decision tree (DT) models. The ensemble model is a machine-learning technique that combines several models to create a superior predictive model, enhancing accuracy, robustness, and overall performance. This addresses data complexity, reduces overfitting, and offsets the limitations of single models by aggregating diverse predictions.

XGBoost has high performance and speed, being an advanced implementation of gradient boosting techniques that excel in classification and regression tasks.

MLPs are a class of feedforward artificial neural networks that model complex nonlinear relationships by learning through multiple layers of nodes.

DT models, on the other hand, are straightforward, tree-structured models that split data into branches at decision nodes, making them easy to interpret and useful for classification and regression.

By combining these models, the ensemble approach harnesses their respective strengths, thereby improving the overall accuracy and reliability of predicting pedestrian crossing intentions.

The final stage of the process tracks the movements of pedestrians at each waiting area and predicts their crossing intentions so as to proactively activate the corresponding signal phase and reduce crossing violations.

### Pedestrian Crossing Prediction

It is an object of the system to accurately predict the crossing direction of pedestrians at intersections while considering the variations in the geometric design of the waiting area, particularly the ramps. The quantity and size of ramps vary across intersections, with the most challenging scenario being a single small ramp serving multiple crosswalks at the studied intersections. In such cases, the location of the pedestrian or VRU does not clearly reveal the intended crossing direction.

To achieve a generalized prediction system, the ensemble model trained on crossing and non-crossing events at different geometric locations with relevant designs is utilized. The features collected per prediction case are divided into eight features of the pedestrian and two main features of the geometric design of the waiting area, as shown in FIG. 4.

These ten features are described in Table 1 below, which shows statistical measures giving parameters of each feature's distribution. Each feature has a type (continuous or discrete), indicating whether it is measured in units like degrees or pixels or grouped into categories. The count is the total number of observations, which is the same for all features. The mean represents the average value, indicating the central tendency of each feature. The standard deviation (std) provides an understanding of how spread out the values are around the mean. The minimum (min) and maximum (max) values show the lowest and highest observed values in the dataset.

The features of the pedestrian include body pose (heading of the main body and distance between the shoulder keypoints), head pose, walking direction, whether the pedestrian is located in the start crossing zone of any crosswalk, distance to each crosswalk (typically, there are two crosswalks at each waiting area), and the difference in distance between them. The geometric design of the waiting area features the distance between the start of the crosswalks and the number of ramps.

TABLE 1
Summary statistics of the variables used in the crossing intention prediction model

| Variable | Type | Count | Mean | Std | Min | Max |
|---|---|---|---|---|---|---|
| VRU body pose angle (body | Continuous (degree) | 140618 | 85 | 24.9 | 0 | 173 |
| pose angle) | | | | | | |
| Shoulder length of VRU | Continuous (pixel) | 140618 | 26.8 | 17.4 | 0 | 126 |
| (shoulder length) | | | | | | |
| VRU head pose (head pose | Discrete (0: front, | 140618 | — | — | 0 | 2 |
| angle) | 1: right, 2: left) | | | | | |
| VRU walking heading | Continuous (degree) | 140618 | 130.6 | 81.7 | 0 | 341.0 |
| direction (VRU heading | | | | | | |
| direction) | | | | | | |
| Is VRU in any start-crossing | Discrete | 140618 | — | — | 0 | 2 |
| zone? (startcrossing zone) | (0: no, | | | | | |
| | 1: in start crossing | | | | | |
| | zone of crosswalk A, | | | | | |
| | 2: in start crossing | | | | | |
| | zone of crosswalk B) | | | | | |
| Relative distance between | Continuous (pixel) | 140618 | 288.3 | 200.9 | 2.24 | 787.1 |
| VRU and start of crosswalk A | | | | | | |
| (dA) | | | | | | |
| Relative distance between | Continuous (pixel) | 140618 | 195.6 | 186.8 | 1 | 947.9 |
| VRU and start of crosswalk B | | | | | | |
| (dB) | | | | | | |
| Absolute difference between | Continuous (pixel) | 140618 | 302.1 | 174.4 | 0.08 | 640.9 |
| VRU_to_cwA and | | | | | | |
| VRU_to_cwB | | | | | | |
| (difference(dA, dB)) | | | | | | |
| Distance between start of | Continuous (feet) | 140618 | 16.2 | 9.9 | 5 | 33 |
| crosswalks (distance | | | | | | |
| between CWs) | | | | | | |
| Number of ramps | Discrete | 140618 | — | — | 1 | 2 |

The system has a primary objective to accurately predict the crossing direction of pedestrians at intersections by analyzing the extracted features from each pedestrian and considering the variations in the geometric design of the waiting area, particularly the ramps. The quantity and size of ramps vary across intersections, with the most challenging scenario being a single small ramp serving multiple crosswalks at the studied intersections. In such cases, the location of the pedestrian does not clearly reveal the intended crossing direction.

To achieve a generalized crossing prediction model, the AI system supports an ensemble model in which the ensemble model is trained on various pedestrian crossings at different geometric locations with relevant designs. The features collected per prediction case are divided into eight features of the pedestrian and two main features of the geometric design of the waiting area, as shown in FIG. 4. The pedestrian features include data defining, or derived from, body pose (main body heading and distance between the shoulder keypoints), head pose (categorized as looking right, left, or front), walking direction, whether the VRU is located in any of the start-crossing zones, distance to the start of each crosswalk (typically, there are two crosswalks at each waiting area), and the difference in distance between them. The geometric design features of the waiting area include the distance between the start of the crosswalks and the number of ramps.

The VRU-CrossSafe system for crossing intention prediction in real-world scenarios addresses some problems. The two main challenges that were addressed are:
first, dealing with shade and different lighting conditions during the day that negatively affect the pose estimation; and
second, achieving real-time processing while dealing with visual feeds, keeping track of all of the pedestrians' movements, and extracting important information about each one.

### Shades and Lighting Conditions

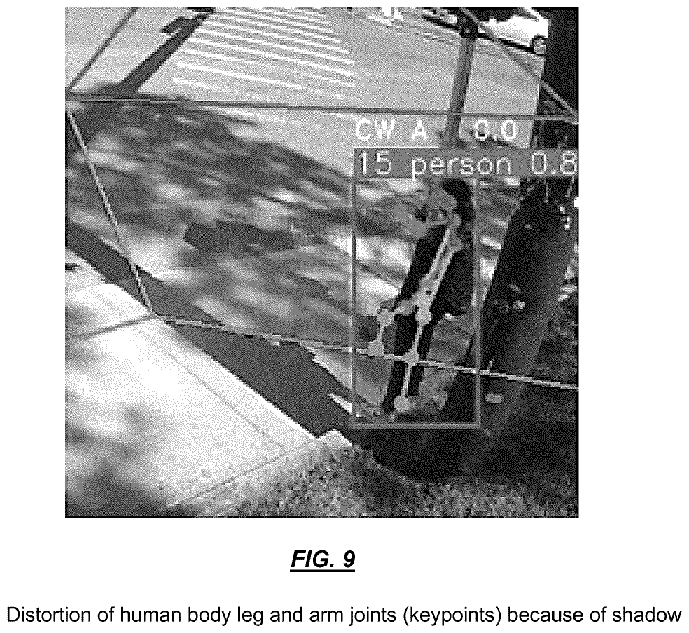

Changing the lighting conditions because of clouds and the presence of shades of trees, traffic poles, and others affects the accuracy of pose estimation. The most disturbed keypoints of the pedestrian human body are the lower keypoints in addition to the arm keypoints (elbows and wrists) as shown in FIG. 9.

Accordingly, only selected keypoints, as shown in FIG. 4, of the pedestrian's body are included in the decision-making process of predicting crossing intentions. The head pose is estimated based on the angles between the eyes and the nose (θf1, θf2), and the body pose is estimated based on the angles between the shoulders and the waist (θb1, θb2) and the distance between the shoulders (d). For each camera view, the head and body pose are used to estimate the direction in which each pedestrian is looking.

### Real-Time Processing

Real-time processing is achieved through seamless integration of computer vision algorithms and deep learning models for VRU detection, tracking, and pose estimation. While YOLOv8 is a real-time object detection, incorporating pose estimation significantly increases computational demand. Therefore, region-based pose estimation is applied, where pose estimation will only be active when the VRU is in the waiting area to determine the VRU's head and body poses. When the VRU is on the crosswalk, the pose estimation is stopped to decrease the latency; however, trajectory tracking will continue.

To further increase the processing speed, the video resolution was reduced from 2K to (864×648). The framework also benefits from using Python libraries, which are less demanding of processing resources.

A notable example is the use of the OpenCV library for output video visualization, as opposed to the PIL library. OpenCV processes images as NumPy arrays, allowing image cropping through array slicing, which makes it faster than PIL. These efforts have boosted the processing speed from 7 FPS on average to 33 FPS enabling real-time video processing.

### Phase Extension Checker

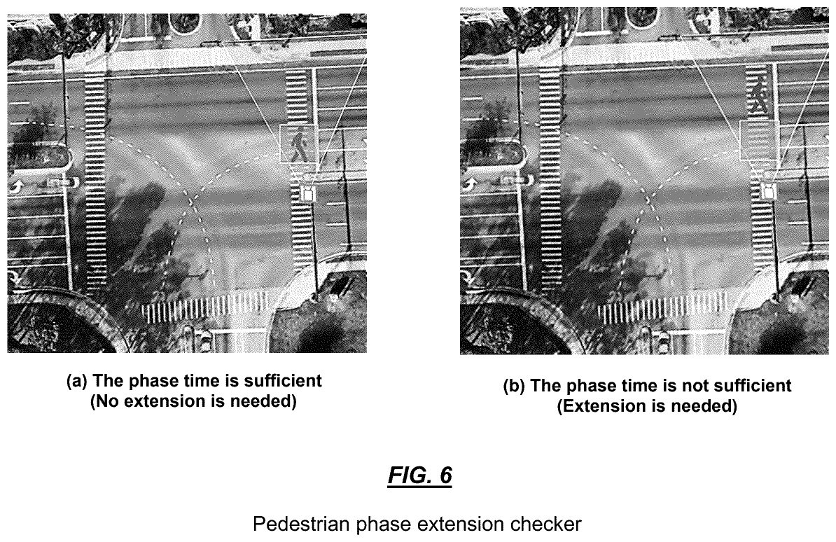

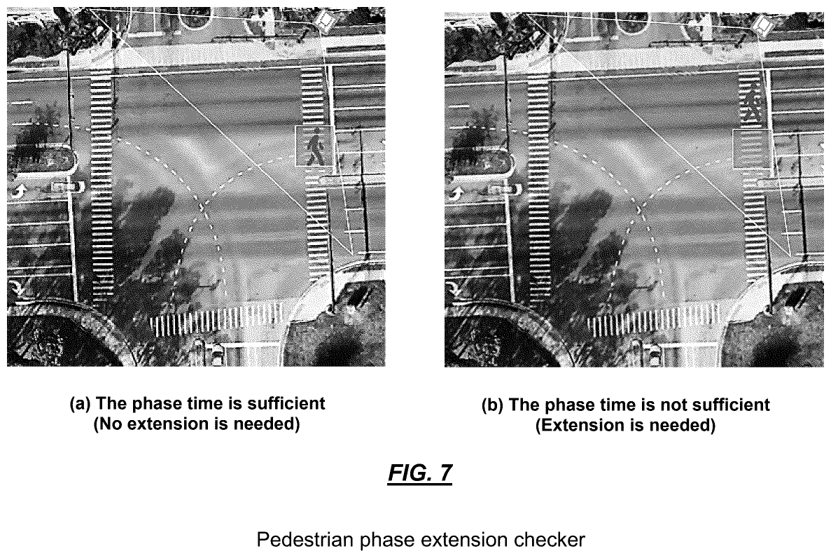

Some pedestrians or VRUs may require additional time to cross for various reasons, such as a disability that necessitates the use of a wheelchair or the accompaniment of children. In such cases, they might need an extension of the pedestrian phase signal time. Accordingly, by monitoring pedestrians' movements on the crosswalk, a virtual checkpoint, positioned just before the midpoint of the crosswalk, serves as an extension checker, as best seen in FIGS. 6 and 7.

Responsive to the pedestrian crossing command, the controller of the intersection typically will initiate a pedestrian crossing phase during which the crosswalk being used by the pedestrian has a WALK signal or other indicator that is displayed for a predetermined period of time selected by the traffic engineer to permit a typical pedestrian to cross the street. If a pedestrian or VRU did not reach that checkpoint within that predetermined time period, which is calculated based on the crosswalk distance and a walking speed of 3.75 ft/sec, which lies within the recommended walking speed according to the U.S. Manual of Uniform Traffic Control Devices, then an extended period of time difference can be added to the pedestrian phase signal time, providing additional crossing time for the pedestrian to ensure safety and a better crossing experience.

### VRU Crossing Intentions

VRU crossing intentions can be categorized as crossing (and which crosswalk is to be crossed), or not crossing.

For a more nuanced understanding, intentions have been classified into multiple behaviors, such as walking, standing, starting, and stopping, and employing observer ratings to determine intention levels. Typically, pedestrians are labeled as crossing or not crossing, with intermediate categories reflecting specific behaviors such as head-turning to observe traffic. Pedestrian behavior in a dataset spanning different countries and lighting conditions has been labeled and found common patterns such as “standing, looking, and crossing” or “moving, looking, and crossing”.

Tracking VRU motion at intersections is a crucial step in identifying VRU actions, where deep learning (DL) has revolutionized the monitoring of pedestrian actions through enhanced detection and tracking with cameras. The evolution from initial models that utilized handcrafted features to advanced DL architectures represents major advancements that can contribute to enhancing the safety of VRUs.

### Object Detection

The present invention preferably employs deep learning-based pedestrian detection.

Table 2 below shows an ablation study of the mentioned SOTA DL models for object detection.

TABLE 2
Ablation Experiments of the proposed models
on the COCO test-dev 2017 dataset

| Model | mAP | Params (M) | FPS |
|---|---|---|---|
| Yolov7 | 51.4 | 36.9 | 161 |
| Yolov8 | 53.9 | 68.2 | 283 |
| Mask R-CNN | 39.8 | 44.6 | 5 |
| DETR | 42.0 | 41 | 28 |

The performance metrics of Table 2 are based on the mean average precision (mAP), the number of parameters, in millions (M), of each model, and the frames per second (FPS). The models were tested on the COCO test-dev dataset by Microsoft. COCO (Common Objects in Context) is a large-scale image recognition dataset designed for object detection, segmentation, and captioning tasks. It contains more than 330,000 images with more than 2.5 million object instances labeled with bounding boxes and masks.

Based on the comparison of the human detection deep learning models, the preferred model for the preferred embodiment is YOLOv8, which achieves state-of-the-art performance and is very fast compared to the other deep learning computer vision-based models. YOLOv8 is therefore the best choice for real-time object detection.

### Object Tracking

VRU tracking models and algorithms address provide for multiobject tracking (MOT).

Tables 3 and 4 present the performances of the proposed tracking algorithms on the DanceTrack and MOT17 datasets. DanceTrack, by Sun et al. (2022), is a dataset for multihuman tracking that focuses on two main characteristics.

First, human subjects have a uniform appearance, making them difficult to distinguish from one another.

Second, they exhibit complex and varied motion patterns with frequent exchanges of relative positions.

The MOT17 dataset described in Milan et al., “MOT16: A benchmark for multi-object tracking”, arXiv preprint arXiv:1603.00831 (2016) is a benchmark dataset for multiple object tracking that contains videos of people and vehicles in various scenes captured from different camera views. It includes fourteen (14) video sequences with high levels of occlusion, crowd density, and complex motion patterns, along with ground-truth annotations for multiple-object tracking evaluation. The dataset is commonly used for evaluating the performance of tracking algorithms.

For the DanceTack ablation study, the performance metrics were based on HOTA, DetA, AssA, MOTA, and IDF1. These metrics are used to measure the accuracy of multi-object tracking algorithms. The greater the values of these parameters are, the better the model's performance.

TABLE 3
Comparison of proposed trackers on the MOT17 test dataset

| Tracker: | OC-SORT | ByteTrack | SORT | DeepSORT | MOTR | StrongSORT |
|---|---|---|---|---|---|---|
| FPS: | 29.0 | 29.6 | 113 | 13.8 | 7.5 | 7.5 |

TABLE 4
Comparison of proposed trackers on the DanceTrack test dataset

| Tracker | HOTA↑ | DetA↑ | AssA↑ | MOTA↑ | IDF1↑ |
|---|---|---|---|---|---|
| OC-SORT | 54.6 | 80.4 | 40.2 | 89.6 | 54.6 |
| Byte Track | 47.3 | 71.6 | 31.4 | 89.5 | 52.5 |
| SORT | 47.9 | 72.0 | 31.2 | 91.8 | 50.8 |
| DeepSORT | 45.6 | 71.0 | 29.7 | 87.8 | 47.9 |
| MOTR | 54.2 | 73.5 | 40.2 | 79.7 | 51.5 |

The definitions of the terms above are as follows:
Higher-order tracking accuracy (HOTA): This metric accounts for the correspondence between the predicted and actual tracks.
Detection accuracy (DetA): This metric compares correctly detected objects to the total ground truth objects.
Assignment accuracy (AssA): Assesses the accuracy of matching detections to track assignments.
Multiple Object Tracking Accuracy (MOTA): A comprehensive metric for overall detection and tracking accuracy that factors in errors.
ID F1 score (IDF1): Evaluates the accuracy of individual track identification using the harmonic mean of F1 scores.

For the MOT17 dataset, the evaluation is based on the FPS only to test the real-time ability of the tracking algorithms, which is one of the main considerations.

Based on the numerical results in Tables 3 and 4, the preferred embodiment employs OC-SORT as the preferred tracking model. OC-SORT excels in scenarios with occlusions and nonlinear movements while offering real-time tracking capabilities. Despite SORT and StrongSORT being competitive models, OC-SORT is more suitable because it is faster than StrongSORT and better than SORT in terms of the performance metric.

### Human VRU Pose Estimation

The VRU pose estimation of the system involves the analysis of visual feedback to estimate the body movements of individuals. By breaking down the human body into seventeen (17) main key points (joints), such as the shoulders, elbows, wrists, hips, knees, and ankles (FIG. 4), pose estimation models can estimate the location of the main joints of the human body, which helps in understanding the orientation and body language of VRUs, which is crucial in estimating their actions and predicting their intentions. The integration of VRU pose estimation in road safety mechanisms is a proactive approach to safeguarding VRUs, contributing significantly to the reduction of traffic-related injuries and fatalities.

The human pose estimation of VRU tracking integrates keypoint detection to capture nuanced body language, which is crucial for estimating crossing intentions.

While trajectories provide basic movement patterns, the inclusion of body gestures such as leg movements and body turns enriches intention prediction models. Gaze and head orientation may be relevant. Techniques such as CNNs for video-based keypoint detection, activity recognition, and identifying distracted drivers have improved the depth of pose estimation applications. LSTM models paired with Bayesian inference can predicted crossing intentions with good accuracy, while pose sequence analysis can yield reliable kinematic data. Zhu et al., “Dual-position features fusion for head pose estimation for complex scene”, Optik 270, 169986 (2022) described a method that improves head pose estimation accuracy in challenging scenes. Zhang et al., “Pedestrian crossing intention prediction at red-light using pose estimation”, IEEE transactions on intelligent transportation systems 23(3), 2331-2339 (2021) described pose estimation for predicting red-light crossing behavior. Zhong et al., “Pedestrian motion trajectory prediction with stereo-based 3D deep pose estimation and trajectory learning”, IEEE access 8, 23480-23486 (2020) described enhanced trajectory prediction accuracy via 3D space tracking. Marginean et al., “Understanding pedestrian behaviour with pose estimation and recurrent networks”, 2019 6th International Symposium on Electrical and Electronics Engineering (ISEEE), IEEE, pp. 1-6 (2019) described use of deep learning for classifying pedestrian behavior, indicating potential improvements through extended sequence analysis.

The system of the invention may employ any of these or any other suitable pose estimation to derive equivalent pose data for the detected VRU.

### Sensors Employed in Pedestrian Research Studies

Various facets of pedestrian behavior prediction and vehicular interaction exist that enhance traffic safety and management, focusing on integrating technology such as cameras or LiDAR for real-time data collection.

Pedestrian speed measurement may done using drone footage, and LiDAR technology may also be used to capture pedestrian and traffic behavior for the development of automated driving systems and safety assessment, respectively. However, cameras continue to be the favored sensors for pedestrian-related research due to their superior resolution and color information, and are the data sensors used in the preferred embodiment of the present invention.

### Summary of Crossing Intention Detection

The preferred embodiment of the system of the invention employs the following for estimating the crossing intentions of VRUs at intersections:
Data collection: The most sophisticated option for VRU detection and tracking is to use video feeds from cameras at intersections.
VRU Detection and Pose Estimation: YOLOv8 is used in the preferred embodiment for real-time processing. YOLOv8 has separate models for human detection and pose estimation, which are preferably efficiently merged.
VRU Tracking: OC-SORT is used in the preferred embodiment because it substantially achieves superb results in most performance metrics.

By leveraging the array of methodologies, models, and advanced deep learning algorithms available, coupled with a concerted effort to refine code for efficiency and ensure quality data acquisition, the system is capable of accurately estimating VRU crossing intentions at intersections in real time. This involves applying state-of-the-art object detection and tracking models and a deep dive into the nuances of code optimization to speed up processing times without sacrificing accuracy. Precise data collection with cameras is essential because it underpins the system that predicts VRU behavior. Integrating these data with advanced technology creates a system that proactively responds to VRU movements and crossing intentions, improving safety at urban intersections.

### Example: Data Collection

For the Example, data was collected from three large four-leg intersections with notable VRU activity, providing a comprehensive dataset.

Intersections were chosen based on their proximity to significant shopping areas, schools, or educational institutions to encompass a broad spectrum of VRU categories. These three intersections are in Florida, United States, where the specified intersections are Alafaya Trail and Central Florida Blvd (intersection 1), Lake Mary Blvd and US 17-92 (intersection 2), and N Alafaya Trail and Science Dr (intersection 3). A total of 1026 VRUs were collected from these sites, embodying a diverse array of VRUs (Table 5), including pedestrians (adults, youths, and people with disabilities); users of nonmotorized vehicles (such as cyclists, scooter riders, skateboarders, and rollerblade users); and individuals on powered two-wheelers (PTWs), such as electric scooters, bikes, and skateboards.

TABLE 5
Types of VRUs captured in the collected video dataset

| | |
|---|---|
| Pedestrian | 70% |
| non-motorized VRU | 25% |
| PTW VRU | 5% |

### Data Collection for Training

Data from the first two intersections were gathered to train ML models for predicting VRU crossing intentions, and subsequent testing was conducted using the collected data from the third intersection. At each intersection, four cameras were strategically placed to cover all waiting areas and crosswalks, as shown in, e,g, FIG. 1E.

The hardware setup, shown in FIG. 2, featured IP cameras operating at 2K resolution and 20 frames per second, offering a 103-degree viewing angle. Cameras were positioned on 10-foot tripods, while other necessary equipment, such as battery stations and Wi-Fi routers, was housed in a waterproof enclosure, with the battery supporting up to 20 hours of operation. This setup enabled the collection of 7 hours of videos during the day from each camera on a weekday, capturing an average of 42 VRUs per hour at the first intersection. At the second intersection, the data collection lasted 5 hours on a weekday, during which an average of 20 VRUs were recorded per hour. The aggregate number of VRUs recorded at these two locations, which served as the dataset for training the models, totaled 437.

### Data Collection for Testing

The ability of the system to predict VRU crossing intentions was tested at different intersections to evaluate the generalizability and robustness of the system.

The same setup for data collection at the first two intersections was used at the third intersection. During a weekday, the camera recorded 10 hours of footage, observing an average of 59 VRUs per hour utilizing the crosswalks at the intersection. Notably, the testing dataset includes data gathered during both daytime and nighttime conditions, which is crucial for assessing the model's effectiveness throughout the entire day, particularly during night hours when pedestrian accidents are more likely. This dataset for the testing phase included a total of 589 VRUs, revealing a wider variety of VRU types than did the training dataset.

### Results

Numerical and visual outcomes of the VRU-CrossSafe system for predicting VRU crossing intentions at intersections highlight unique captured instances.

The hardware configuration for this system includes an Intel Core i7-7820X CPU with 16 cores clocked at 3.60 GHz paired with an Nvidia RTX 2080Ti GPU and 32 GB of RAM.

### Results of Training and Validation

In total, 140,618 data points were prepared for training, where each data point represented the crossing prediction per frame of the VRU, where the features of the VRU were obtained from the extracted trajectories and pose estimation.

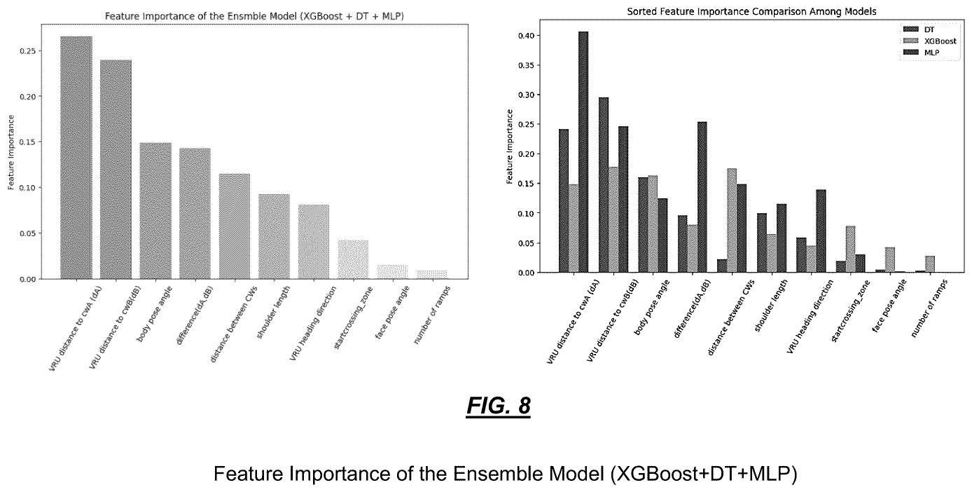

The geometric features and VRU crossing intentions were manually labeled across all the data points. The ensemble model attained a 99.93% accuracy on the training dataset, as shown in FIG. 8, demonstrating the significance of different features for predicting VRU crossings. Among all the features considered, the MLP assigns greater importance to the relative distance between the VRU and the start of the crosswalking than do the other ML models. DT gives lower importance to the geometric design of the waiting area (distance between the starting points of the crosswalks and the number of ramps), while XGBoost considers almost all of the features to be significant. Despite the varied importance assigned by each ML model, the ensemble, which combines all of these machine learning models, considers the most critical attributes for crossing prediction to be the VRUs' relative positions to the beginning of the two crosswalks (A and B), followed by the body pose angle indicating the VRU's posing direction and the distance between the starting points of the crosswalks.

### Numerical Results

For rigorous testing of the ensemble model, data from an intersection different from the two used for training were assessed.

Impressively, the ensemble model demonstrated excellent performance metrics, as shown in Table 6, achieving an accuracy of 94.67% and a low false alarm rate on the test dataset, which included 11 different types of VRUs, including normal pedestrians, distracted pedestrians, children, elderly, mothers with infants in strollers, and people on various types of smaller conveyances, including various bicycles or skateboards (powered or not), or roller skates. This notable degree of accuracy over all these types of VRUs attests to the thorough data preparation and the model's ability to predict crossing intentions for various types of VRUs, demonstrating its superiority over other similar models in the context of real-time VRU crossing prediction.

TABLE 6
Performance metrics of the ensemble model on the test dataset

| | | | | False | False |
|---|---|---|---|---|---|
| | | | | Positive | Negative |
| Model | Accuracy | Precision | Recall | (FP) Rate | (FN) rate |
| Ensemble | 94.67% | 88.24% | 95.63% | 6.72% | 4.37% |
| Model | | | | | |

The performance of the proposed predictive system is also compared to the existing signal timing strategies to highlight the potential time savings. The results are divided into three categories based on the type of event such as (a) time saving by eliminating dual push button (b) time saving by dropping a phase call for departed pedestrian and (c) wait time reduction for vehicle gap out in times of low vehicle demand. The results are show in the Table 7. It can be noticed that the average time saved for eliminating push button is 30.5 seconds per cycle while for dropping a phase for a departed pedestrian, the time saved is 32.7 seconds. Additionally, for scenario (c), the waiting time of a pedestrian is also reduced by 32.9 seconds on average. Thus, by prioritizing pedestrian phase during low traffic volume, such as AM/PM off-peak hours, it is possible to accommodate pedestrians earlier in the cycle thereby increasing compliance and reducing the chances of jaywalking. On average this system has the potential to reduce the cycle lengths by 15% without compromising pedestrian safety.

TABLE 7
Average Time Saving based on proposed system

| | Cycle Length | Cycle Length | Average Time | Reduction in |
|---|---|---|---|---|
| Scenario | Before (s) | After (s) | Saved (s) | cycle length (%) |
| (a) Eliminate dual | 205.8 | 175.3 | 30.5 | 15 |
| push button | | | | |
| (b) Drop phase call | 205.3 | 172.5 | 32.7 | 17 |
| for departed ped | | | | |
| | Waiting Time | Waiting Time | Average Time | Reduction in |
| | Before (s) | After (s) | Saved (s) | cycle length (%) |
| (c) Gap out vehicle | 87.2 | 54.2 | 32.9 | 14 |
| phase | | | | |

### Visual Results of Special Cases

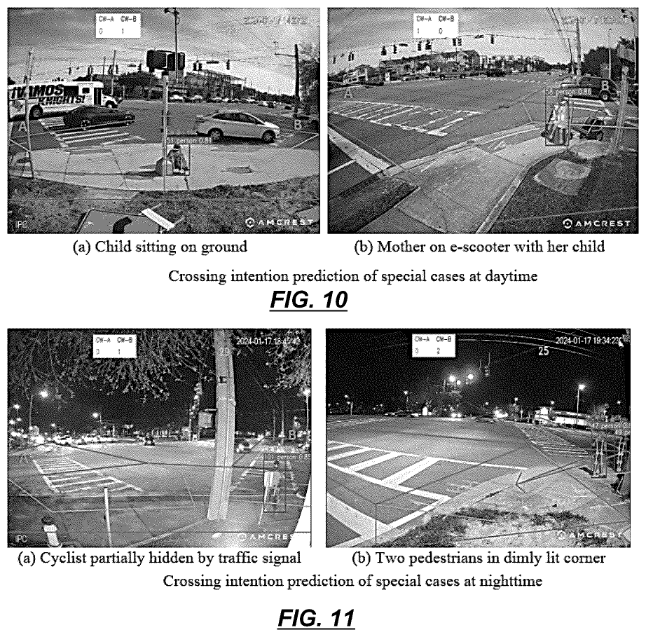

FIGS. 10 and 11 illustrate scenarios in which the ensemble model successfully predicted the crossing intentions of VRUs, highlighting the need for this system to be implemented at many intersections for real-time VRU crossing prediction, thereby enabling proactive activation of the corresponding phase signal. These examples also highlight some of the challenging cases that the prediction model faced in determining the crossing intentions of VRUs, especially since the training dataset lacks a significant number of instances involving children or VRUs using motorized and non-motorized modes.

FIG. 10 depicts two daytime cases: the first (FIG. 10a) shows a child sitting on the ground, intending to cross at crosswalk B without pressing the push button, and waiting for more than 5 minutes before deciding to violate the signal and jaywalk. The second (FIG. 10b) shows the case of a mother riding an e-scooter with her child in a mobile cart. Any crossing violation by such VRUs can expose them to dangerous situations involving oncoming traffic.

FIG. 11 shows two scenarios occurring at night, where the training dataset had no nighttime instances. Despite these challenges, the VRU-CrossSafe system successfully predicted the crossing intentions of bicyclists, even though a traffic sign partly obscured their body and accurately predicted the crossing intentions of two pedestrians in the dimly lit corner of the waiting area.

### Other Aspects

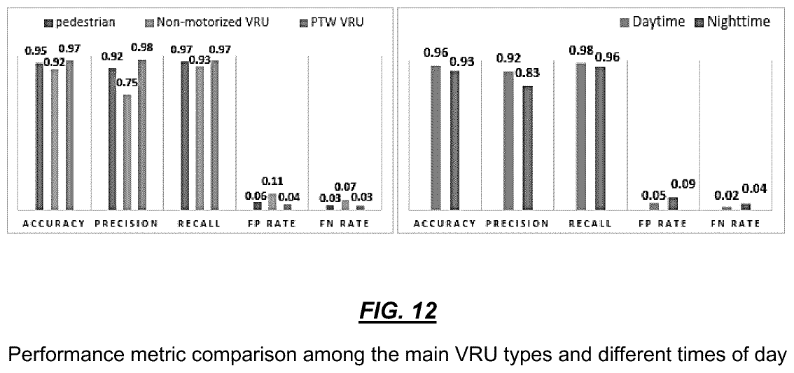

Analysis using the detailed test dataset has led to significant results, as shown in FIG. 12.

The two subfigures compare the performance metrics across the three main categories of VRUs: pedestrians, nonmotorized vehicle users, and PTW users. Notably, the results reveal that the prediction accuracy for pedestrians and PTW users is considerably greater than that for users of nonmotorized micromobility. This distinction suggests that pedestrians and PTW users may exhibit more predictable or consistent behaviors when approaching and crossing streets, which aligns better with the system's predictive models.

Additionally, the analysis indicates a slight decline in performance metrics between daytime and nighttime predictions. During daylight hours, the system demonstrated greater accuracy and reliability in predicting VRU crossing intentions and less accuracy under nighttime conditions. This decline in performance during the night suggests that current intersection lighting may not be adequate for optimal system performance, potentially leading to decreased predictability of VRU movements.

These findings suggest enhancing intersection lighting and improving intersection infrastructure to increase VRU compliance during crossing events to ensure that safety measures and predictive capabilities remain robust across all times of day and for all types of VRUs.

### Extension Checker Results

According to an aspect of the invention, the system determines whether a VRU needs more time to cross the road and reach the other side safely. This is done by tracking the VRUs.

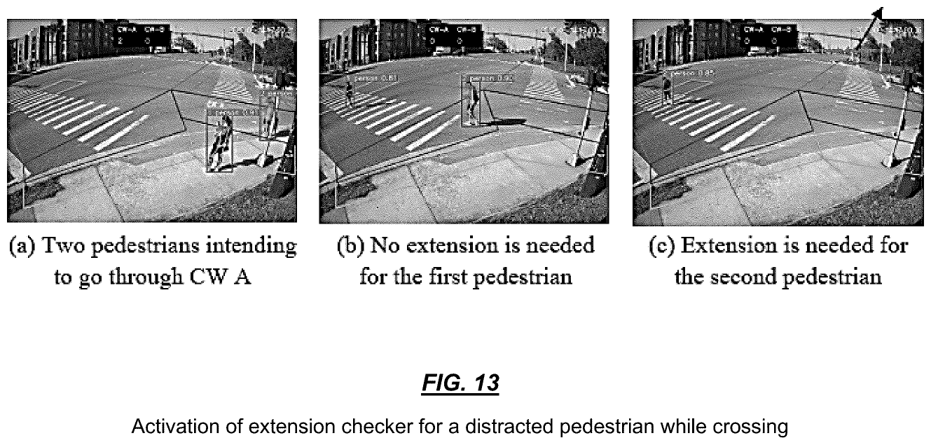

FIG. 13 shows an empirical example of two pedestrians who were predicted to go through crosswalk A. After they started crossing, the VRU-CrossSafe system tracked them to ensure that the time given to finish crossing was sufficient.

For the first pedestrian, the pedestrian reached the extension checkpoint within the expected time, which was 8 seconds based on the set walking speed of 3.75 ft/s at that intersection.

The second pedestrian took 10 seconds to reach the extension checker checkpoint. Thus, the pedestrian needed 4 additional seconds (2 seconds to compensate for the first half and the other 2 seconds for the other half of the crosswalk). It was observed that this pedestrian was using a mobile phone while waiting and crossing, which made the VRU distracted, and more time was needed to finish crossing comfortably.

Results have shown that estimating pedestrian crossing intentions in real-time reached an average accuracy of 94.67%. This enables the activation of the pedestrian crossing phase without the need of a push-button as well as ensuring their safety against approaching vehicles by tracking pedestrians while crossing.

### Overall Analysis of Testing Dataset

Although the testing dataset was larger and more varied, encompassing various types of VRUs and including nighttime scenarios unlike the training dataset, the VRU-CrossSafe system demonstrated reliable real-time crossing predictions. It achieved an excellent average accuracy of 94.67% across four cameras at the test intersection compared to that of other methods.

The system also achieved real-time processing, estimating VRU crossing intentions at an average processing speed of 33 FPS without skipping any frames. The system has among the highest processing speeds for video feed analysis in systems designed to predict VRU crossing intentions.

VRU-CrossSafe has proven effective in managing predictions both during the day and at night, with tests confirming its efficiency under various weather conditions, such as light rain and overcast conditions, thus demonstrating its robustness and reliability. Moreover, the system's ability to filter out false calls significantly enhances signal performance and results in more efficient traffic flow at intersections.

The integration of an extension checker is advantageous for VRUs needing more time to cross intersections safely and comfortably.

Due to the accuracy of these real-time crossing predictions, this system is appropriate for deployment by many entities focused on enhancing VRU safety at intersections. Its adaptability can be extended from intersections to different types of junctions, including midblocks, due to its flexibility in conforming to the geometric design of crosswalk areas. Moreover, through Infrastructure-to-Vehicle (I2V) communication, the VRU-CrossSafe system can transmit crossing predictions to approaching autonomous and connected vehicles, prompting them to exercise caution when crossing VRUs and preventing potential accidents.

The VRU-CrossSafe system represents a substantial advancement in road safety, as it targets the protection of VRUs at intersections. By harnessing state-of-the-art deep learning and computer vision techniques for VRU detection, tracking and pose estimation, YOLOv8 and OC-SORT, the system automatically predicts VRU crossing behavior without relying on push buttons, thereby reducing the likelihood of signal violations, errors, and potential conflicts with vehicles by activating the corresponding signal phase and reducing crossing violations.

The system, when installed at three four-legged intersections, preferably uses four roadside cameras to monitor VRU movements, incorporating an extension checker for those needing more crossing time. With a high accuracy of 94.67%, testing on a different intersection capturing 589 pedestrians and other vulnerable road users during both daytime and nighttime, and processing average processing speed of 33 FPS, the VRU-CrossSafe system sets a new benchmark, outstripping other methods in real-time processing for crossing prediction.

This system also can employ I2V communications to further reduce accidents by alerting drivers to VRUs' presence and actions.

Adoption of the VRU-CrossSafe system by transportation departments at intersections or midblock crossings with high VRU volumes is expected to significantly reduce fatalities and injuries among VRUs.

Additionally, automatically activating the pedestrian phase signal for individuals who either forget to press the push button or press both buttons, which can cause unnecessary signal delays, will enhance their trust in and encourage the use of sustainable mobility options.

The system may be adjusted by expanding tests to additional locations or other countries where VRU behavior differs from the VRU behavior of the Example. In addition, integration of the system with the extension checker add-on will allow for adjustment of the set crossing speeds for VRUs, enhancing signal performance. This adjustment can be beneficial because many VRUs utilizing nonmotorized and motorized micromobility vehicles require less time to cross than typical pedestrians, potentially saving significant time for the traffic signal and improving its efficiency.

What is claimed is:

1. A method for controlling a traffic signal directed to vehicles travelling on a roadway to which the traffic signal is directed, said method comprising:
automatically monitoring an area adjacent a pedestrian crossing so as to detect the presence of a pedestrian therein; and
detecting physical parameters of the pedestrian;
determining from the physical parameters of the pedestrian whether there is a likelihood that the pedestrian will cross the pedestrian crossing; and
responsive to a determination that the pedestrian is likely to cross the pedestrian crossing, causing the traffic signal to display a directive to said vehicles so that the vehicles permit the pedestrian to cross the pedestrian crossing.

2. The method of claim 1, wherein the sensing and making of the determination of the pedestrian crossing is performed using a camera generating an electronic signal that comprises video of the pedestrian in said area.

3. The method of claim 1, wherein the making of the determination includes deriving location data and pose data for the pedestrian and making the determination of intent of the pedestrian to cross the pedestrian crossing therefrom.

4. The method of claim 3, wherein the location data includes data identifying the pedestrian crossing and data indicative of a distance of the detected pedestrian therefrom;

5. The method of claim 3, wherein the pose data comprises data indicative of position of a head or upper body of the pedestrian.

6. The method of claim 1, wherein the causing of the traffic signal to display the directive is either performed by direct control of the traffic signal, or by transmitting an electronic signal to a controller that is configured to control the traffic signal responsive to activation of a pedestrian push button and the electronic signal sent to the controller is treated by the controller as a signal from the activation of the pedestrian push button when pushed.

7. The method of claim 1, wherein the making of the determination is by computer system that is either an AI system or a computer provided with programming derived from an AI system, and said AI system was trained to differentiate between pedestrians that cross at the pedestrian crossing and pedestrians that are sensed but do not cross by providing to the AI system historical pose, location and crossing result data comprising:
a number of pedestrian location and pose data sets; and
for each of the pedestrian location and pose data sets, a respective crossing result data indicative of whether the pedestrian crossed the pedestrian crossing.

8. The method of claim 7, wherein the area is also adjacent a second pedestrian crossing, and the AI system has been trained to determine which of the pedestrian crossings the pedestrian is likely to cross.

9. The method of claim 1, wherein, when the traffic signal is a red light directed to traffic that may turn right and enter the pedestrian crossing, and it is determined from the video signal that the pedestrian has entered, or is likely to enter, the pedestrian crossing, the directive displayed by the traffic signal includes a red light signal and a display indicating that vehicles at the red light should not turn right during the red light signal, said display indicating that vehicles at the red light should not turn right during the red light signal being displayed only when it is determined that the pedestrian is in the pedestrian crossing or intends to enter the pedestrian crossing.

10. The method of claim 1, wherein, when the traffic signal is a green light directed to traffic that may turn right and enter the pedestrian crossing, and it is determined from the video signal that the pedestrian has entered, or is likely to enter, the pedestrian crossing, the directive displayed by the traffic signal includes a display indicating that turning vehicles should yield to pedestrians, said display indicating that turning vehicles should yield to pedestrians being displayed only when it is determined that the pedestrian is in the pedestrian crossing or intends to enter the pedestrian crossing.

11. The method of claim 1, wherein the method further comprises
sensing presence of the pedestrian in the pedestrian crossing, and
determining whether the pedestrian crossing the pedestrian crossing is still in the pedestrian crossing during a time period of the display of the directive, and
responsive to the determination that the pedestrian is still in the crossing, extending the time period of the display of the directive for the traffic signal long enough to allow the pedestrian to complete crossing of the pedestrian crossing.

12. The method of claim 1, wherein the method is applied to an intersection having two or more phases of traffic control, and the method further comprises
sensing other pedestrians in at least one area adjacent at least one other pedestrian crossing for which at least one other traffic signal is configured to display directives to vehicles affecting said at least one other pedestrian crossing; and
determining whether pedestrians in the area have an intent to cross the other pedestrian crossing, and
responsive to a determination of the intent, causing the other traffic signal to display a directive against vehicles entering the pedestrian crossing in the pedestrian crossing associated therewith.

13. The method of claim 1, wherein the historical pose, location and crossing result data is provided from an intersection different from where the method is applied.

14. The method of claim 1, wherein the method further comprises
sensing presence of the pedestrian after the controller has been directed to process a pedestrian push button signal; and
directing the controller to cancel the processing of the pedestrian signal responsive to a determination that the pedestrian has left the crosswalk and the area adjacent thereto.

15. The method of claim 1, wherein the determination of the physical parameters of the pedestrian includes estimation of pose data for the pedestrian, and the estimation of the pose data is performed only after a determination that the pedestrian has been in the area for at predetermined period of time, and said estimation of the pose data foe the pedestrian being discontinued when the likelihood that the pedestrian will cross the pedestrian crossing is determined.

16. An electronic system automatically controlling a traffic signal directed to vehicles travelling on a roadway to which the traffic signal is directed with a pedestrian crossing across said roadway, said system comprising:
a controller controlling the traffic signal so as to selectively display thereon directives to at least some of the vehicles on the roadway;
an automatic pedestrian crossing system connected with the controller, said automatic pedestrian crossing system having
at least one camera having a field of view covering an area at least adjacent to the pedestrian crossing;
a computerized pedestrian monitoring system connected with the camera and receiving therefrom an electronic video signal comprising a series of frames of the field of view of the camera;
said pedestrian crossing system determining from the electronic video signal if a pedestrian is present in the area adjacent the pedestrian crossing, and responsive to a determination that a pedestrian is present in the area for a period of time of at least three seconds, deriving pose estimation data from the video signal corresponding to a pose of the pedestrian in the area;
said pedestrian crossing system having a neural network trained to differentiate between poses of pedestrians that intend to cross the pedestrian crossing and pedestrians in the area that do not have said intent to cross the pedestrian crossing, said neural network processing the pose estimation data and deriving therefrom a determination whether or not the pedestrian has an intent to cross the pedestrian crossing;
said pedestrian crossing system being connected with the controller and transmitting to the controller an electronic command responsive to a determination that the pedestrian has an intent to cross the pedestrian crossing;
responsive to receiving the electronic command, the controller being configured to cause the traffic signal to enter an active pedestrian crossing state wherein the traffic signal displays a traffic directive configured to avoid conflict of some of the vehicles with pedestrians in the pedestrian crossing.

17. The electronic system according to claim 15, wherein the neural network is trained by providing thereto historical pose, location and crossing result data comprising:
a number of pedestrian location and pose data sets; and
for each of the pedestrian location and pose data sets, a respective crossing result data indicative of whether the pedestrian crossed the pedestrian crossing.

18. The electronic system according to claim 15, wherein the field of view also covers at least a portion of the pedestrian crossing, and the controller is configured to cause the traffic signal to enter the active pedestrian crossing state for a predetermined period of time, and the pedestrian crossing system monitoring the video signal so as to determine whether the pedestrian is in the pedestrian crossing when the predetermined period of time has elapsed, and, responsive to a determination that the pedestrian is still in the pedestrian crossing, issuing an electronic command to extend the active pedestrian crossing state of the traffic signal for an additional period of time.

19. The electronic system according to claim 15, wherein an additional pedestrian crossing is adjacent said area, and the determination of the intent of the pedestrian includes an identification of which of the pedestrian crossings the pedestrian has the intent to cross.

20. The electronic system according to claim 15, wherein the pedestrian crossing system monitors the video signal so as to determine whether the pedestrian actually enters the pedestrian crossing after the command to enter the active pedestrian crossing state of the traffic signal is sent to the controller, and responsive to a determination that the pedestrian has not entered the pedestrian crossing, transmitting a cancel command causing the controller to terminate the active pedestrian crossing state.

21. The electronic system according to claim 15, wherein the system includes at least one blank out display configured to display a NO TURN ON RED display, said system causing the blank out display to display the NO TURN ON RED display only when the computerized pedestrian monitoring system determines that a pedestrian intends to enter, or is in, the pedestrian crossing and the pedestrian crossing is an approach crosswalk for a lane of traffic to which the controller is presenting a red light traffic signal.

22. The electronic system according to claim 15, wherein the system includes at least one blank out display configured to display a TURNING VEHICLES YIELD display, said system causing the blank out display to display the TURNING VEHICLES YIELD display only when the computerized pedestrian monitoring system determines that a pedestrian intends to enter or is in the pedestrian crossing and the pedestrian crossing is a departing crosswalk for a lane of traffic to which the controller is presenting a green light traffic signal.

23. A computer accessible memory device storing thereon data corresponding to instructions causing a computer system to perform the method of claim 1.
